(19) United States
(12) Patent Application Publication
(10) Pub. No.: US 2023/0000835 A1
(43) Pub. Date: Jan. 5, 2023

(54) LYSINE-SPECIFIC HISTONE DEMETHYLASE INHIBITORS FOR THE TREATMENT OF MYELOPROLIFERATIVE NEOPLASMS

(71) Applicant: Imago Biosciences, Inc., San Carlos, CA (US)

(72) Inventor: Hugh Y. RIENHOFF, JR., San Carlos, CA (US)

(21) Appl. No.: 17/805,130

(22) Filed: Jun. 2, 2022

Related U.S. Application Data
(63) Continuation of application No. PCT/US2020/063773, filed on Dec. 8, 2020.
(60) Provisional application No. 62/945,609, filed on Dec. 9, 2019.
(60) Provisional application No. 63/121,461, filed on Dec. 4, 2020.

(51) Int. Cl. A61K 31/4192; A61P 35/02

(52) U.S. Cl. CPC A61K 31/4192; A61P 35/02

(57) ABSTRACT

Disclosed herein are methods for treating or preventing myeloproliferative neoplasms in a subject in need thereof, and for effecting specific clinically relevant endpoints, comprising administering a therapeutically effective amount of an LSD1 inhibitor.

This application is a continuation of International Application No. PCT/US2020/063773, filed Dec. 8, 2020, which claims the benefit of U.S. Provisional Application No. 62/945,609, filed Dec. 9, 2019, and U.S. Provisional Application No. 63/121,461, filed Dec. 4, 2020, which are hereby incorporated by reference as if written herein in their entireties.

Myeloproliferative neoplasms (MPN), a disease category that includes polycythemia vera (PV), essential thrombocytosis (ET) and myelofibrosis (MF), are a distinct family of hematopoietic disorders caused by somatic mutations acquired by a multipotent hematopoietic stem/progenitor cell resulting in abnormalities in hematologic disturbances in red cell, white cell, and platelet production as well as splenomegaly and constitutional symptoms. The MPNs share common mutations which constitutively alter the normal physiologic signals responsible for hematopoiesis. MPN may present clinically as a benign clonal myeloproliferation but the initiating abnormal stem/progenitor cell is susceptible to new mutations and epigenetic alterations that allow for the rapid evolution to bone marrow failure with myelofibrosis or transformation to acute myelogenous leukemia (AML).

Many MPN patients are asymptomatic at the time of diagnosis. Confounding a definitive diagnosis and prognosis, ET, PV and PMF can masquerade as one another. Common presenting manifestations include fatigue, weight loss, night sweats, fever, dyspnea, and abdominal discomfort due to sometimes massive splenomegaly. The three MPN disorders overlap phenotypically and even share similarities with other myeloid neoplasms. A specific point mutation in JAK2 (JAK2^{V617F}) as well as mutations in calreticulin (CALR) and the thrombopoietin receptor (MPL) are found in 90% of MPN patients. Although the distribution of these mutations is not equal among PV, ET and primary MF PMF), they do not diagnostically define the specific MPN or the prognosis nor are they mutually exclusive. Healthy individuals may carry one of these mutations without developing an MPN, indeed some of these mutations can be carried as germline mutations giving rise to hereditary forms of MPNs. ET, PV and PMF are nevertheless regarded as separate clinical entities each based on a distinct epidemiology, natural history and molecule profile. PV is the most common MPN and would appear to be the phenotypic manifestation mutations in JAK2. PV is the only MPN characterized by erythrocytosis defined as a hematocrit ≥60% and hemoglobin ≥20 gm/dL. ET is characterized by a sustained platelet count of >450,000/μL and occurs predominantly in women. MF, primary or secondary myelofibrosis but sometimes called myelofibrosis with myeloid metaplasia, agnogenic myeloid metaplasia, or primary myelosclerosis, is a chronic inflammatory process in which excess collagen is deposited in bone marrow impairing hematopoiesis in association with marrow fibrosis and extramedullary hematopoiesis.

The major complications arise from cytopenias secondary to bone marrow failure, extramedullary hematopoiesis, principally in the spleen and liver, and evolution to acute myeloid leukemia. To patients, splenomegaly is the most distressing complication of primary myelofibrosis, leading to mechanical discomfort, inanition, splenic infarction, portal and pulmonary hypertension and blood cell sequestration. Both ET and PV are complicated by thrombosis. ET and PV can progress to MF as well as to AML.

Many other somatic mutations found in MPN are also present in myelodysplastic syndrome (MDS) and de novo AML; these include mutations in DNMT3A, IDH1/2, TET2, ASXLI, EZH2, TP53, NF1, NRAS, KRAS, SF3B1, U2AF1, SRSF2 and RUNX1. This shared mutational spectrum contributes to the phenotypic overlap of these disorders as well as influences their natural history including evolution to bone marrow failure or AML.

There is no treatment specific for primary myelofibrosis, essential thrombocythemia or polycythemia vera. Current treatments do not alter the natural history of disease significantly and are thus aimed principally at improving symptoms. Anemia associated with an erythropoietin (EPO) level <100 mU/ml may respond to recombinant EPO therapy but is associated with an increase in hepatosplenomegaly. Prednisone may be effective for patients with evidence of active inflammation or autoimmune disease. Hyperuricemia is managed with allopurinol. The nonselective JAK1/2 inhibitor ruxolitinib is approved for intermediate 1 and 2 and high risk MF patients and high-risk PV patients. Ruxolitinib is effective in alleviating constitutional symptoms and reducing spleen size or volume by 35% in approximately 50% of patients. Ruxolitinib prolonged survival and lowered the JAK^{V617F} allele burden in high-risk patients with primary MF (PMF). Anemia is exacerbated by ruxolitinib in some patients but thrombocytopenia, even if severe, may be improved. Ruxolitinib is effective only while the drug is administered; symptoms will recur when the drug is stopped. Fibrosis in the marrow is not affected and ruxolitinib has no impact on the mutation burden. Thalidomide at doses of 50 to 100 mg/day in combination with prednisone is effective in improving anemia and thrombocytopenia in approximately 60% of primary myelofibrosis patients and reducing spleen size in approximately 20%. Interferon-α at low-doses to reduce splenomegaly can be effective in the early course of the illness but can cause cytopenias. Pegylated interferon can produce molecular remissions in PV and reverse myelofibrosis in PMF in a minority of patients. Hydroxycarbamide has a low incidence of acute toxicity but causes marrow suppression and is leukemogenic. Low-dose alkylating agents can reduce organomegaly, reverse marrow fibrosis, and improve blood counts but only occasionally has durable effects; alkylating agents can cause severe bone marrow suppression and are leukemogenic. The only potentially curative treatment is allogeneic bone marrow transplantation indicated for patients younger than 65 years of age with intermediate-2 or high DIPSS score who have a matched donor. Five-year survival from stem cell transplant averages is approximately 50%.

Epigenetic modifications of DNA such as methylation of cytosine or post-translational modifications of histones such as methylation and acetylation influence gene expression by altering chromatin structure. Changes in gene expression patterns have the potential to alter the phenotype of a given cell. Mutations in DNMT3A and TET2 are associated with changes in the normal methylation patterns of cytosine in DNA while mutations in JAK2, EZH2 and ASXLI alter the methylation, acetylation and phosphorylation state of histones: both of these classes of changes alter the patterns of normal gene expression programs. Mutations in genes coding for proteins influencing the epigenetic state of the cells suggest that targeting the enzymatic function of such proteins may selectively eliminate malignant stem/progenitor clones and/or restoring their normal phenotype.

Lysine-specific demethylase 1 (LSD1, also known as KDM1A) is an enzyme that removes mono- and dimethyl groups from histone (H) H3 at critical lysines (K), K4 and K9 (Shi et al., 2004). Methylation of histone H3K4 and H3K9 is a post-translational modification associated with changes in rates of gene. By virtue of altering the local state of chromatin, LSD1 is an epigenetic regulator of gene expression. The lysine (K) sites on histone H3 and the degree of methylation on those sites (1, 2 or 3 methyl groups) are associated with specific functions, e.g., enhancers and super-enhancers are characterized by H3K4me1 marks, whereas H3K4me2 is more often found in the proximal promoters and enhancers of actively transcribed genes.

LSD1 is localized to three general regions of the genome: enhancers and super-enhancers, proximal promoters, and internal regions of transcription units through the agencies of proteins that bind DNA directly, generally TFs. Many TFs, both activators such as V-Myb Avian Myeloblastosis Viral Oncogene Homolog (MYB) and steroid hormone receptors, as well as repressors such as growth factor independence 1 transcription repressor (GFI1), recruit LSD1 to specific genomic locations. LSD1 is part of a larger protein complex, containing, e.g., Co-RE 1 silencing transcription factor (CoREST) or nucleosome remodeling and histone deacetylase (NuRD), which dictate the cell-specific chromatin remodeling. These complexes may also include DNMT1 and histone deacetylases 1, 2 and 3 (HDAC1, 2, and 3) activities, all of which contribute to maintaining or modifying the epigenetic state at that genomic site. Thus, an important property of LSD1 beyond its own enzymatic activity is its function as a scaffold for other epigenetic enzymes that are co-recruited to genomic sites. Among the many histone demethylases, LSD1 uniquely employs flavin adenine dinucleotide (FAD) to oxidatively remove one or two methyl groups in the process producing H2O2 and formaldehyde. As such, FAD is an essential co-factor for LSD1 activity. The other 33 histone lysine demethylases, the Jumonji types, employ an iron-dependent mechanism to remove methyl groups from histone lysines.

LSD1 is an essential gene; loss of LSD1 activity leads to early embryonic lethality. The protein is also needed for regulating the balance between self-renewal and proliferation. A conditional in vivo LSD1 knockdown (KD) using a doxycycline-inducible short hairpin LSD1 (shLSD1) established LSD1 as a central regulator of hematopoietic stem cells (HSCs) and myeloid progenitor cells. LSD1 KD resulted in profound but reversible thrombocytopenia, neutropenia and anemia; monocyte numbers were increased. LSD1 KD for 27 days led to an increase in circulating multipotent progenitors (MPPs) and HSCs with a concomitant down-regulation of chemokine (C-X-C motif) receptor 4 (CXCR4) without affecting the size of the dormant HSC pool. Impaired self-renewal was observed in long term HSCs 12 weeks following LSD1 excision using an inducible Cre system (Mx1Cre mice×Lsd1fl/fl mice), consistent with LSD1 inhibition driving differentiation.

LSD1 plays a key role in regulating the progression from pluripotency to terminal differentiation. LSD1 is recruited to “high confidence” promoters and super-enhancers of genes essential for normal development by the “master” transcription factors octamer-binding transcription factor 4 (OCT4), SRY (sex determining region Y)-box 2 (SOX2), Nanog and the co-activator Mediator. Though not essential for maintenance of the embryonic stem cell (ESC) state, as part of the NuRD complex, LSD1 “decommissions” enhancers of genes directing the pluripotency program allowing ESC differentiation. LSD1 is essential for the complete shutdown of the ESC gene expression program as cells transition to more differentiated cell states. The role LSD1 plays in the ESC is phenomenologically similar to the essential role LSD1 plays during myeloid hematopoiesis, in which enhancers active in HSCs generating a stem-cell gene expression signature are also “decommissioned”, allowing commitment of progenitors to specific myeloid lineages. Enhancers essential for terminal differentiation in lineage-specific progenitor cells are poised for activation by H3K4me1 marks while promoters are characterized by progressive methylation of H3K4 culminating in H3K4me3. Enhancer H3K27 acetylation locks in transcriptional activation and lineage commitment. Consistent with the need for stable H3K4 methylation during differentiation, LSD1 expression decreases dramatically as myeloid differentiation proceeds to terminal cell states. The LSD1 enzyme sits at the apex of myeloid hematopoiesis. LSD1 prevents myeloid differentiation in stem and myeloid progenitor cells but is down-regulated as cells commit to specific myeloid lineages (erythroid, granulocytic, and megakaryocytic). The inhibition of LSD1 in acute myeloid leukemia cells causes a loss of stem cell potential (clonogenicity) and a concomitant induction of differentiation to a more mature monocytic immunophenotype. In mouse models of myeloproliferative neoplasm, treatment with LSD1 inhibitors reduces the mutant progenitor cell population consistent with the role LSD1 plays in sustaining the self-renewal phenotype.

As a key factor in regulating myeloid maturation, LSD1 is suitable as a target for a variety of myeloproliferative neoplasms. There are three major myeloproliferative neoplasms that may be treated with an LSD1 inhibitor: polycythemia vera, essential thrombocythemia, primary myelofibrosis (or myelofibrosis secondary to PV and ET); other MPNs are disclosed below and may also be treated by the methods disclosed herein. Other MPNs include All begin as clonal disorders as a consequence of somatic mutations occurring in hematopoietic stem/progenitor cells. The clinical overlap among these related diseases is mirrored by their shared genetic spectrum of somatic mutations including mutations in JAK2, DNMT3A, MPL, CALR, and ASXL1. In mouse models of myelofibrosis (Jak2^{6V17F} and Mpl^{W515L}), inhibition of LSD1 causes a significant improvement in five parameters of disease: reduction in platelets, reduction in splenomegaly, reduction in red cell count, resolution of marrow fibrosis and reduction in mutant cell burden.

Among BCR-ABL-negative myeloproliferative neoplasms, primary myelofibrosis and post-PV/ET myelofibrosis (PPV-MF and PET-MF) are associated with the highest degree of morbidity and mortality, including progressive bone marrow (BM) fibrosis and resultant BM failure. Although the JAK inhibitor ruxolitinib is now approved for the treatment of MF-associated splenomegaly and systemic symptoms, JAK inhibitor therapy does not reduce the population of JAK2-mutant cells in MPN patients. The limited ability of JAK inhibition to induce clinically meaningful molecular responses in MPN patients underscores the need for the development of more effective therapies for these JAK kinase/STAT-dependent malignancies.

Recent studies have shown that the lysine-specific histone demethylase, LSD1 (KDM1A), participates in the balance in hematopoietic stem/progenitor cells between proliferation and differentiation in vivo by influencing state-specific gene expression patterns. In physiologic hematopoiesis, LSD1 is essential for normal myeloid differentiation affecting the erythroid, megakaryocytic and granulocytic lineages but not the monocytic/dendritic lineage. Small molecule inhibitors of LSD1 have shown promising results in preclinical models of acute myeloid leukemia (AML) and solid cancers and have recently entered clinical trials in AML. However, the role and requirement for LSD1 in the pathogenesis of MPNs and the therapeutic targeting of LSD1 in MPN is an area of current investigation.

WO 2012/107498 discloses the use of certain LSD1 inhibitors to treat the Philadelphia chromosome negative myeloproliferative disorders essential thrombocythemia, myelofibrosis, and polycythemia vera. US 2016/0257662 and US 2016/0237043 disclose compounds that inhibit LSD1. US 2019/0070172 discloses the utility of these compounds and others in the treatment of myeloproliferative neoplasms including ET, MF, and PV.

There remains, however, a need for potent LSD1 inhibitors with demonstrated ability to treat myelofibrosis and other myeloproliferative neoplasms, and attendant symptoms, and achieve specific, clinically relevant endpoints in the treatment of myelofibrosis and other myeloproliferative neoplasms, while avoiding serious side effects such as severe thrombocytopenia.

### BRIEF DESCRIPTION OF THE DRAWINGS

FIG. 1 shows the change in spleen volume of patients treated with LSD1 inhibitor Compound 1, from day 0 to day 84 of treatment.

FIG. 2 shows the change in MPN-10 scores of patients treated with LSD1 inhibitor Compound 1, from day 0 to day 84 of treatment.

FIG. 3 compares treatment with LSD1 inhibitor Compound 1 to the Best Available Treatment (BAT), in terms of changes in spleen volume response (SVR) and total symptom score (TSS), from day 0 to day 84 of treatment.

FIG. 4 shows the change in inflammatory cytokine S100A9 at week 12 in the course of treatment with LSD1 inhibitor Compound 1

FIG. 5 shows the change in inflammatory cytokine RANTES at week 12 in the course of treatment with LSD1 inhibitor Compound 1

FIG. 6 shows the change in inflammatory cytokine IL-8 at week 12 in the course of treatment with LSD1 inhibitor Compound 1

FIG. 7 shows the change in circulating growth factor VEGF at week 12 in the course of treatment with LSD1 inhibitor Compound 1

FIG. 8 shows the change in circulating growth factor PDGF-BB at week 12 in the course of treatment with LSD1 inhibitor Compound 1.

FIG. 9 is a schematic representation of a therapeutic theory of LSD1 inhibition by Compound 1.

FIG. 10 shows the percent of F-cells in six patients treated with LSD1 inhibitor Compound 1.

FIG. 11 shows absolute change in (a) MPN SAF TSS and (b) spleen volume from (i) Day 0 to (ii) 12 weeks.

FIG. 12 shows the progress of treatment for a representative patient. (a) daily dose of LSD1 inhibitor, mg; (b) spleen size, cm; (c) symptoms score; (d) platelets (left scale, k/uL) and hemoglobin (right scale); (e) WBC and neutrophils; and (f) fatigue score (10=worst).

### DETAILED DESCRIPTION

Provided herein is a method for treating a myeloproliferative neoplasm in a subject in need thereof, the method comprising administering a therapeutically effective and non-deleterious amount of an LSD1 inhibitor.

Also provided is a method for suppressing proliferation of malignant myeloid cells in a subject in need thereof, the method comprising administering a therapeutically effective and non-deleterious amount of an LSD1 inhibitor.

Also provided is a method for reducing the concentration of one or more protein growth factors secreted by bone marrow cells that activate one or more cell types that secrete reticulin and collagen in a subject in need thereof, the method comprising administering a therapeutically effective and non-deleterious amount of an LSD1 inhibitor.

In certain embodiments, the one or more protein growth factors is/are chosen from a platelet-derived growth factor, vascular endothelial growth factor, transforming growth factor beta 1 and platelet factor 4 (aka CXCL4).

In certain embodiments, the bone marrow cells that activate one or more cell types that secrete reticulin and collagen are megakaryocytes.

In certain embodiments, the one or more cell types that secrete reticulin and collagen is chosen from stromal cells and/or bone marrow-resident fibroblasts and/or myofibroblasts.

Also provided is a method for reducing the concentration of one or more protein growth factors secreted by bone marrow cells that impair the function of bone marrow osteoclasts to reduce the amount of bone marrow osteosclerosis in a subject in need thereof, the method comprising administering a therapeutically effective and non-deleterious amount of an LSD1 inhibitor.

In certain embodiments, the bone marrow cells that that impair the function of bone marrow osteoclasts to reduce the amount of bone marrow osteosclerosis in the subject are megakaryocytes.

Also provided is a method for reducing reticulin and collagen bone marrow fibrosis in a subject in need thereof, the method comprising administering a therapeutically effective and non-deleterious amount of an LSD1 inhibitor.

Also provided is a method for reducing plasma levels of one or more inflammatory cytokines in a subject in need thereof, the method comprising administering a therapeutically effective and non-deleterious amount of an LSD1 inhibitor.

Also provided is a method for reducing the malignant cell burden measured by the mutant allele frequency of myeloid cells in a subject in need thereof, the method comprising administering a therapeutically effective and non-deleterious amount of an LSD1 inhibitor.

Also provided is a method for eliminating malignant myeloid cells in a subject in need thereof, the method comprising administering a therapeutically effective and non-deleterious amount of an LSD1 inhibitor.

Also provided is a method for reducing a pathologically elevated red blood cell mass in a subject in need thereof, the method comprising administering a therapeutically effective and non-deleterious amount of an LSD1 inhibitor.

Also provided is a method for reducing abnormal spleen size or volume in a subject in need thereof, the method comprising administering a therapeutically effective and non-deleterious amount of an LSD1 inhibitor.

Also provided is a method for reducing the amount of extramedullary hematopoiesis in a subject in need thereof, the method comprising administering a therapeutically effective and non-deleterious amount of an LSD1 inhibitor.

Also provided is a method for improving the quality of life (QOL) measured by validated patient-reported QOL assessments in a subject in need thereof, the method comprising administering a therapeutically effective and non-deleterious amount of an LSD1 inhibitor.

Also provided is a method for reducing the constitutional symptoms of myelofibrosis measured by validated patient-reported symptom assessment forms in a subject in need thereof, the method comprising administering a therapeutically effective and non-deleterious amount of an LSD1 inhibitor.

Also provided is a method for extending the life span of a subject with myelofibrosis in need thereof, the method comprising administering a therapeutically effective and non-deleterious amount of an LSD1 inhibitor.

Also provided is a method for delaying or preventing the progression of myelofibrosis to acute myeloid leukemia in a subject in need thereof, the method comprising administering a therapeutically effective and non-deleterious amount of an LSD1 inhibitor.

Also provided is a method for reducing platelet counts in a subject in need thereof, the method comprising administering a therapeutically effective amount of an LSD1 inhibitor.

Also provided is a method for reducing bone marrow cellularity to age-adjusted normocellularity with fewer than 5% blast cells in a subject in need thereof, the method comprising administering a therapeutically effective and non-deleterious amount of an LSD1 inhibitor.

Also provided is a method for maintaining the bone marrow blast count or reducing the bone marrow blast count to <5% in a subject in need thereof, the method comprising administering a therapeutically effective and non-deleterious amount of an LSD1 inhibitor.

Also provided is a method for increasing hemoglobin to >100 g/L in a MF patient, comprising administering a therapeutically effective amount of an LSD1 inhibitor.

Also provided is a method for reducing the frequency of thrombosis and hemorrhage in a subject in need thereof, the method comprising administering a therapeutically effective and non-deleterious amount of an LSD1 inhibitor.

Also provided is a method for reducing the frequency of transfusions of red blood cells in a subject in need thereof, the method comprising administering a therapeutically effective and non-deleterious amount of an LSD1 inhibitor.

Also provided is a method for a) reducing the hematocrit in a male patient with PV to <45% or reducing the hematocrit in a female patient with PV to ≤42% b) reducing the hemoglobin level in a PV patient to <160 g/L, and/or c) decreasing red cell mass in a PV patient to ≤5.2M/mL, either comprising administering a therapeutically effective and non-deleterious amount of an LSD1 inhibitor.

In certain embodiments of each of the above methods, the LSD1 inhibitor is N-[(2S)-5-{[(1R, 2S)-2-(4-fluorophenyl) cyclopropyl]amino}-1-(4-methylpiperazin-1-yl)-1-oxopentan-2-yl]-4-(1H-1,2,3-triazol-1-yl)benzamide, bis-tosylate salt

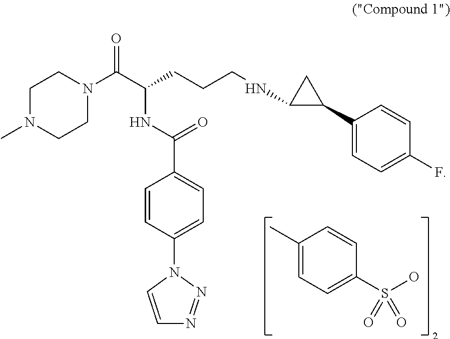

Also provided herein is a method of treating a myeloproliferative neoplasm and achieving a platelet count of about 50×10⁹ to about 100×10⁹ platelets/L in a subject, comprising:
administering a starting dose of 0.5 mg/kg/d Compound 1;
after about one week, assessing the subject's platelet count;
if platelet count is ≥90×10⁹ platelets/L and the % platelet reduction is <50% from previous visit, add 0.2 mg/kg/d Compound 1 to the daily dose;
if platelet count is ≥90×10⁹ platelets/L and the % platelet reduction is ≥50% from previous visit, add 0.1 mg/kg/d Compound 1 to the daily dose;
if platelet count is between 40×10⁹ platelets/L and 89×10⁹ platelets/L, maintain the current daily dose of Compound 1;
if platelet count is between 25×10⁹ platelets/L and 39×10⁹ platelets/L, decrease the current mg/kg daily dose of Compound 1 by 25%;
if platelet count is <25×10⁹ platelets/L, withhold dosing until platelets return to >50×10⁹ platelets/L, then administer Compound 1 at 50% of the dose that was administered when platelet count fell below 25×10⁹ platelets/L; and
optionally, repeating the platelet count assessment and dose adjustment steps approximately weekly until the subject's platelet count is about 50×10⁹ to about 100×10⁹ platelets/L.

In certain embodiments, the subject in need has a myeloproliferative neoplasm.

In certain embodiments, the myeloproliferative neoplasm is myelofibrosis (MF).

In certain embodiments, the myelofibrosis is chosen from primary myelofibrosis (PMF), post-PV myelofibrosis (PPV-MF), and post-ET myelofibrosis (PET-MF).

In certain embodiments, the myelofibrosis is primary myelofibrosis (PMF).

In certain embodiments, the myeloproliferative neoplasm is polycythemia vera (PV).

In certain embodiments, the myeloproliferative neoplasm is essential thrombocythemia (ET).

In certain embodiments, said subject has, or the subject's malignant myeloid cells have, a mutation in one or more genes chosen from Janus Kinase 2 (JAK2), myeloproliferative leukemia virus oncogene (MPL) and calreticulin (CALR).

In certain embodiments, the method further comprises the step of determining whether said subject has mutations in one or more genes chosen from Janus Kinase 2 (JAK2), myeloproliferative leukemia virus oncogene (MPL) and calreticulin (CALR).

In certain embodiments, the therapeutically effective and non-deleterious amount of Compound 1 is an amount sufficient to maintain in the subject with myelofibrosis a platelet count of about 50×10⁹ to about 100×10⁹ platelets/L, or an amount otherwise described below. In certain embodiments, the therapeutically effective and non-deleterious amount of Compound 1 is an amount sufficient to maintain in the subject a platelet count of about 50×10⁹ to about 75×10⁹ platelets/L.

In certain embodiments, the therapeutically effective and non-deleterious amount of Compound 1 is an amount sufficient to maintain in the patient with essential thrombocythemia a platelet count below 400×10⁹, or an amount otherwise described below.

In certain embodiments, the therapeutically effective and non-deleterious amount of Compound 1 is an amount sufficient to maintain in the patient with PV a platelet count of about 150×10⁹ to about 250×10⁹ platelets/L, or an amount otherwise described below.

In certain embodiments, the therapeutically effective and non-deleterious amount of Compound 1 is about 0.5 mg/kg/d to about 1.5 mg/kg/d.

In certain embodiments, the therapeutically effective and non-deleterious amount of Compound 1 is about 0.7 mg/kg/d to about 1.2 mg/kg/d.

In certain embodiments, the therapeutically effective and non-deleterious amount of Compound 1 is about 40 mg to about 100 mg per day.

In certain embodiments, the therapeutically effective and non-deleterious amount of Compound 1 is about 50 mg to about 85 mg per day.

In certain embodiments, the subject is administered a starting dose of 0.5 mg/kg/d Compound 1, then, after one week:
if platelet count is ≥90×10⁹ platelets/L and the % platelet reduction is <50% from previous visit, the subject's dose is adjusted to add 0.2 mg/kg/d Compound 1 to the daily dose;
if platelet count is ≥90×10⁹ platelets/L and the % platelet reduction is ≥50% from previous visit, the subject's dose is adjusted to add 0.1 mg/kg/d Compound 1 to the daily dose;
if platelet count is between 40×10⁹ platelets/L and 89×10⁹ platelets/L, the daily dose of Compound 1 is maintained;
if platelet count is between 25×10⁹ platelets/L and 39×10⁹ platelets/L, the subject's dose is adjusted to decrease the current mg/kg daily dose of Compound 1 by 25%;
if platelet count is <25×10⁹ platelets/L, withhold dosing until platelets return to >50×10⁹ platelets/L, then the subject's dose is adjusted to administer Compound 1 at 50% of the dose that was administered when platelet count fell below 25×10⁹ platelets/L; and
optionally, approximately weekly throughout the course of therapy, repeating the platelet count assessment and dose adjustment steps until the subject's platelet count is about 50×10⁹ to about 100×10⁹ platelets/L.

Also provided is a method of treating a myeloproliferative neoplasm in a subject in need thereof wherein the subject has a mutant allele, said method comprising:
administering to the subject an amount of N-[(2S)-5-{[(1R, 2S)-2-(4-fluorophenyl) cyclopropyl]amino}-1-(4-methylpiperazin-1-yl)-1-oxopentan-2-yl]-4-(1H-1,2,3-triazol-1-yl)benzamide, bis-tosylate salt

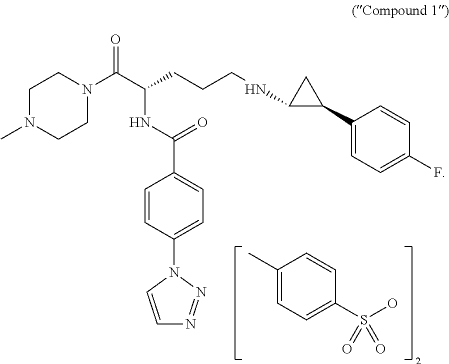

In certain embodiments, the mutant allele is an allele of one or more genes chosen from Janus Kinase 2 (JAK2), such as JAK^{V617F}, myeloproliferative leukemia virus oncogene (MPL), such as MPL^{W515K}, and calreticulin (CALR), such as CALR^{52b_del}, CALR^{K38SNCX}, or CALR^{KKRK374X}.

In certain embodiments, the mutant allele is an allele of one or more genes chosen from chosen from DNMT3A, IDH1/2, TET2, ASXLI, EZH2, TP53, NF1, NRAS, KRAS, SF3B1, U2AF1, SRSF2, RUNX1, CBL, ZBTB33, PRPF8, CNTN5, FREM2, MAP1B, and GPR183.

In certain embodiments, the mutant allele is one or more of ASXL1^{HHCHREAA630X}, ASXL1^{-642X}, ASXL1^{Q780*}, ASXL1^{R693}, ASXL1^{-884X*}, ASXL1^{-642X}, ASXL1^{QLL695HX} and ASXL1^{Q768*}.

In certain embodiments, the mutant allele is an allele of the gene Biorientation Of Chromosomes In Cell Division 1 Like 1 (BOD1L1).

In certain embodiments, the the mutant allele is one or more of BOD1L1^{S1623C}, BOD1L1^{E1612K}, BOD1L1^{K1136N}, BOD1L1^{R1074W}, BOD1L1^{Y812C}, BOD1L1^{E289K}, and BOD1L1^{R508S}.

### Abbreviations and Definitions

To facilitate understanding of the disclosure, a number of terms and abbreviations as used herein are defined below as follows:

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

As used herein, the term “about,” as used herein when referring to a measurable value such as an amount of a compound, dose, time, temperature, and the like, is meant to encompass variations of 20%, 10%, 5%, 1%, 0.5%, or even 0.1% from the specified amount.

As used herein, a “therapeutically effective amount” of a drug is an amount of drug or its pharmaceutically acceptable salt that eliminates, alleviates, or provides relief of the disease for which it is administered, or the symptoms of the disease.

As used herein, a “non-deleterious amount” of a drug is an amount is an amount of drug or its pharmaceutically acceptable salt that does not produce dose-limiting toxicity or side effects. One example of such toxicity/side effect is anemia (hemoglobin <8 grams/dL), severe thrombocytopenia (platelet count <25 k/uL) or severe granulocytopenia (absolute neutrophil count <0.5 k/uL).

As used herein, a “subject in need thereof” is a human or non-human animal that exhibits one or more symptoms or indicia of a disease.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.). When n is set at 0 in the context of “0 carbon atoms”, it is intended to indicate a bond or null.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reaction of a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reaction of the appropriate compound, in the form of the free base, with the appropriate acid.

The compounds disclosed herein can exist as polymorphs and other distinct solid forms such as solvates, hydrates, and the like. A compound may be a polymorph, solvate, or hydrate of a salt or of the free base or acid.

The term “myeloproliferative neoplasm” (MPN) refers to blood cancers that occur when the body makes too many white or red blood cells, or platelets as a consequence of somatic mutations that activate the hormone signaling pathways that control the production of these types of blood cells. They are “clonal diseases of hematopoietic stem cells” given that the neoplastic cells arise from a single mutant clone arising from bone marrow cells (Campregher et al. Rev Bras Hermatol Hemoter. 2012; 34(2):150-5). MPNs include polycythemia vera (PV), myelofibrosis including primary myelofibrosis (PMF, including, in certain embodiments, both the prefibrotic/early stage and the overt fibrotic stage) and post-PV/ET myelofibrosis (PPV-MF and PET-MF), essential thrombocythemia (ET), chronic neutrophilic leukemia (CNL), chronic eosinophilic leukemia, not otherwise specified (CEL-NOS), and chronic myeloid leukemia (CML), as well as other unclassifiable MPNs. For a more thorough discussion of MPNs and related myeloid neoplasms and acute leukemia, as well as diagnostic criteria for PV, ET, PMF, and other MPNs, see Arber et al. “The 2016 revision to the World Health Organization classification of myeloid neoplasms and acute leukemia”, Blood 2016, 127(20):2391-2405. For a thorough discussion of myelofibrosis diagnostic and response criteria, see Tefferi A et al., “Revised response criteria for myelofibrosis: International Working Group-Myeloproliferative Neoplasms Research and Treatment (IWG-MRT) and European LeukemiaNet (ELN) consensus report,” Blood, 122(8):1395-98 (2013).

The following abbreviations may be used throughout the specification and have the meanings assigned.

| Abbreviation | Definition |
|---|---|
| <, ≤, >, ≥, | less than, less than or equal to, greater than, greater than or |
| | equal to |
| ± | plus or minus |
| AE | adverse event |
| AML | acute myeloid leukemia |
| BCR-ABL | breakpoint cluster region-Abelson |
| ° C. | degrees Centigrade |
| CALR | calreticulin |
| CD | cluster of differentiation |
| cGMP | current Good Manufacturing Practices |
| CoREST | Co-repressor for RE1-silencing transcription factor |
| CXCL | chemokine (C-X-C Motif) ligand |
| CTCAE | Common Terminology Criteria for Adverse Events |
| CV | co-efficient of variation |
| D, d | day |
| DLT | dose limiting toxicity |
| DNA | deoxyribonucleic acid |
| DNMT | DNA-methyltransferase |
| DSMC | Data Safety Monitoring Committee |
| ELN | European Leukemia Network |
| EMH | extramedullary hematopoiesis |
| EOS | eosinophils |
| EPO | erythropoietin |
| ET | essential thrombocythemia |
| FAD | flavine adenine dinucleotide |
| Free base of | N-[(2S)-5-{[(1R,2S)-2-(4-fluorophenyl)cyclopropyl]amino}-1-(4- |
| Compound 1 | methylpiperazin-1-yl)-1-oxopentan-2-yl]-4-(1H-1,2,3-triazol-1- |
| (Compound 2) | yl)benzamide, free base |
| g or gm | gram |
| g/dL | gram per deciliter |
| GFI1 | growth factor independent 1 transcription factor |
| GFP | green fluorescent protein |
| GI | gastrointestinal |
| GLP | good laboratory practice |
| GM-CSF | granulocyte-macrophage colony stimulating factor |
| GMP | Good Manufacturing Practices |
| H | histone |
| Hb | Hemoglobin |
| HDAC | histone deacetylase |
| HSC | hematopoietic stem cell |
| HSCT | hematopoietic stem cell transplant |
| IC | inhibitory concentration |
| ICH | International Conference on Harmonization |
| IL | interleukin |
| Compound 1 | N-[(2S)-5-{[(1R,2S)-2-(4-fluorophenyl)cyclopropyl]amino}-1-(4- |
| | methylpiperazin-1-yl)-1-oxopentan-2-yl]-4-(1H-1,2,3-triazol-1- |
| | yl)benzamide, bis-tosylate salt |
| indels | insertions and deletions |
| IWG-MRT | International Working Group for Myelofibrosis Research and |
| | Treatment |
| JAK | Janus Kinases |
| K | lysine |
| KD | knockdown |
| KDM1A | lysine demethylase 1A |
| Kg | kilogram |
| L | liter |
| LDH | lactate dehydrogenase |
| LIC | leukemia initiating cell |
| LPLV | last patient last visit |
| LSD1 | lysine-specific demethylase 1 |
| LSDi | LSD1 inhibition or inhibitors |
| MAO; MAOI | monoamine oxidase(s); monoamine oxidase inhibitor(s) |
| me; Me | methyl; methylation |
| mg | milligram |
| MF | myelofibrosis |
| MF-SAF | Myelofibrosis Symptom Assessment Form |
| mL | milliliter |
| mL/min | milliliters per minute |
| MPL | myeloproliferative leukemia virus oncogene, thrombopoietin receptor |
| MPN | myeloproliferative neoplasias or neoplasms |
| MPP | multipotent progenitor |
| MPN-SAF TSS | Myeloproliferative Neoplasm Symptom Assessment Form Total Symptom |
| | Score |
| mRNA | messenger RNA |
| ms | milliseconds |
| MYB | V-Myb Avian Myeloblastosis Viral Oncogene Homolog |
| NOAEL | no-observed-adverse-effect-level |
| NURD | nuclear remodeling and histone deacetylase |
| OMIM | On-line Inheritance in Man |
| OPG | osteoprotegerin |
| OS | overall survival |
| PD | pharmacodynamics |
| PET-MF | post-essential thrombocythemia myelofibrosis |
| PK | pharmacokinetics |
| PMF | primary myelofibrosis |
| PPV-MF | post-polycythemia vera myelofibrosis |
| PV | polycythemia vera |
| QD | once daily |
| RBC | red blood cell |
| REST | RE-1 silencing transcription factor |
| RNA | ribonucleic acid |
| SAE | serious adverse event |
| SD | standard deviation |
| Sh | short hairpin |
| SOC | standard-of-care |
| SOX2 | see SRY |
| SRY | (sex determining region Y)-box 2; also known as SOX2 |
| STAT | Signal Transducer and Activator of Transcription |
| Tmax | time to maximum concentration |
| TCP | tranylcypromine |
| TF | transcription factor |
| TPO | thrombopoietin |
| μL | microliter |
| WBC | white blood cell |
| WHO | World Health Organization |

### Formulations

While it may be possible for the compounds disclosed herein to be administered as the raw chemical, it is also possible to present them as pharmaceutical formulations (equivalently, “pharmaceutical compositions”). Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, intraadiposal, intraarterial, intracranial, intralesional, intranasal, intraocular, intrapericardial, intraperitoneal, intrapleural, intraprostatical, intrarectal, intrathecal, intratracheal, intratumoral, intraumbilical, intravaginal, intravesicular, intravitreal, and intramedullary), intraperitoneal, rectal, topical (including, without limitation, dermal, buccal, sublingual, vaginal, rectal, nasal, otic, and ocular), local, mucosal, sublingual, subcutaneous, transmucosal, transdermal, transbuccal, transdermal, and vaginal; liposomal, in cremes, in lipid compositions, via a catheter, via a lavage, via continuous infusion, via infusion, via inhalation, via injection, via local delivery, via localized perfusion, bathing target cells directly, or any combination thereof. Administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound disclosed herein or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as hard or soft capsules, wafers, cachets or tablets each containing a predetermined amount of the active ingredient; as a powder or granules; as a syrup, elixir, solution, or a suspension in an aqueous liquid or a non-aqueous liquid; or as an oil-in-water liquid emulsion, a water-in-oil liquid emulsion, or a compound dispersed in a liposome. The active ingredient may also be presented as a bolus, electuary or paste.

Pharmaceutical preparations that can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated to provide delayed, slowed, or controlled release or absorption of the active ingredient therein. Compositions may further comprise an agent that enhances solubility or dispersability. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Depending on the route of administration, the compounds, or granules or particles thereof, may be coated in a material to protect the compounds from the action of acids and other natural conditions that may inactivate the compounds.

The compounds may be formulated for parenteral administration by injection, e.g., by bolus injection or continuous infusion, either to the body or to the site of a disease or wound. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. To administer the therapeutic compound by other than parenteral administration, it may be necessary to coat the compound with, or co-administer the compound with a material to prevent its inactivation (for example, via liposomal formulation).

It should be understood that in addition to the ingredients particularly mentioned above, the formulations described above may include other agents conventional in the art having regard to the type of formulation in question, for example, those suitable for oral administration may include flavoring agents.

Preferred unit dosage formulations are those containing an effective dose, as herein below recited, or an appropriate fraction thereof, of the active ingredient. In certain embodiments, a formulation disclosed herein is administered once a day. However, the formulations may also be formulated for administration at any frequency of administration, including once a week, once every 5 days, once every 3 days, once every 2 days, once a day, twice or more a day, etc. Such dosing frequency is also maintained for a varying duration of time depending on the therapeutic regimen. The duration of a particular therapeutic regimen may vary from one-time dosing to a regimen that extends for months or years. Dose and dosing regimen are discussed further below.

The amount of active ingredient that may be combined with the carrier materials to produce a single dosage form will vary depending upon the host treated and the particular mode of administration. Similarly, the precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. In addition, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is inflammation, then it may be appropriate to administer an anti-inflammatory agent in combination with the initial therapeutic agent. Alternatively, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). There is even the possibility that two compounds, one of the compounds described herein and a second compound may together achieve the desired therapeutic effect that neither alone could achieve. Alternatively, by way of example only, the benefit experienced by a patient may be increased by administering one of the compounds described herein with another therapeutic agent (which also includes a therapeutic regimen) that also has therapeutic benefit. By way of example only, in a treatment for acute myelogenous leukemia or sickle cell anemia involving administration of one of the compounds described herein, increased therapeutic benefit may result by also providing the patient with another therapeutic agent for sickle cell anemia or for acute myelogenous leukemia. In any case, regardless of the disease, disorder or condition being treated, the overall benefit experienced by the patient may simply be additive of the two therapeutic agents or the two agents may have synergistic therapeutic effects in a patient.

Effective combination therapy may be achieved with a single composition or pharmacological formulation that includes both agents, or with two distinct compositions or formulations, at the same time, wherein one composition includes a compound of the present disclosure, and the other includes the second agent(s). Alternatively, the therapy may precede or follow the other agent treatment by intervals ranging from minutes to months. Administration of the compounds of the present disclosure to a patient will follow general protocols for the administration of pharmaceuticals, taking into account the toxicity, if any, of the drug. It is expected that the treatment cycles would be repeated as necessary.

Specific, non-limiting examples of possible combination therapies include use of compounds disclosed herein with the following agents and classes of agents: agents that inhibit DNA methyltransferases such as decitabine or 5′-aza-cytadine; agents that inhibit the activity of histone deacetylases, histone de-sumoylases, histone de-ubiquitinases, or histone phosphatases such as hydroxyurea; antisense RNAs that might inhibit the expression of other components of the protein complex bound at the DR site in the gamma globin promoter; agents that inhibit the action of Klf1 or the expression of KLF1; agents that inhibit the action of Bcl11a or the expression of BCL11A; and agents that inhibit cell cycle progression such as hydroxyurea, ara-C or daunorubicin; agents that induce differentiation in leukemic cells such as all-trans retinoic acid (ATRA); and JAK inhibitors such as ruxolitinib (Jakafi/Jakavi), fedratinib (Inrebic), cerdulatinib (PRT062070), gandotinib (LY-2784544), lestaurtinib (CEP-701), momelotinib (GS-0387, CYT-387), and pacritinib (SB1518).

Thus, in another aspect, the present invention provides methods for treating diseases or disorders in a human or animal subject in need of such treatment comprising administering to said subject an amount of a compound disclosed herein effective to reduce or prevent said disorder in the subject in combination with at least one additional agent for the treatment of said disorder that is known in the art.

### Compounds

Examples of LSD1-inhibiting compounds which may be used in the methods disclosed herein include the compounds below. Other LSD1 inhibitors are known in the art.

### General Synthetic Methods for Preparing Compounds

In the Examples below and throughout the disclosure, the following abbreviations may be used: PTFE=polytetrafluoroethylene; RM=Reaction Mixture; RH=Relative Humidity; RT=Room Temperature; SM=Starting Material; MeCN=acetonitrile; ClPh=chlorophenol; DCE=dichloroethane; DCM=dichloromethane; DIPE=di-isopropylether; DMA=dimethyl acetamide; DMF=dimethyl formamide; DMSO=dimethylsulfoxide; Et₂O=di-ethyl ether; EtOAc=ethyl acetate; EtOH=ethanol; H₂O=water; IPA=propan-2-ol; i-PrOAc=iso-propyl acetate; MEK=methyl ethyl ketone; MeOH=methanol; MIBK=methyl isobutyl ketone; MTBE=methyl tert-butyl ether; n-BuOAc=n-butyl acetate; n-BuOH=n-butanol; NMP=n-methyl pyrrolidone; n-PrOH=n-propanol; s-BuOAc=s-butyl acetate; t-BuOH=t-butanol; TFA=tri-fluoro acetic acid; THF=tetrahydrofuran; TMP=2,2,4-trimethylpentane; ¹H-NMR=Proton Nuclear magnetic Resonance; DSC=Differential Scanning calorimetry; DVS=Dynamic Vapour Sorption; GVS=Gravimetric Vapour Sorption; HPLC=High Performance Liquid Chromatography; HS=Head Space; HSM=Hot Stage Microscopy; IC=Ion Chromatography; IDR=Intrinsic Dissolution Rate; KF=Karl-Fisher; MAS=Magic Angle Spinning; MDSC=Modulated Differential Scanning calorimetry; PLM=Polarised Light Microscopy; PVM=Particle Vision and Measurement; SCXRD=Single Crystal X-Ray Diffraction; SS-NMR=Solid State Nuclear Magnetic Resonance; TGA=Thermal Gravimetric Analysis; UV=UltraViolet VH-XRPD=Variable Humidity X-Ray Powder Diffraction; VT-XRPD=Variable Temperature X-Ray Powder Diffraction; and XRPD=X-Ray Powder Diffraction. Other abbreviations may be used and will be familiar in context to those of skill in the art.

The invention is further illustrated by the following non-limiting examples. The methods exemplified below may also be extrapolated to compounds disclosed herein. Further methods suitable for use in preparation of examples of the present invention may be found in WO 2015/021128 and WO 2016/130952, the contents of which are hereby incorporated by reference as if written herein in their entireties. Additional LSD1 inhibitors may be prepared by methods disclosed above.

### Intermediate A: (1R,2S)-2-(4-fluorophenyl)-1-methylcyclopropanamine

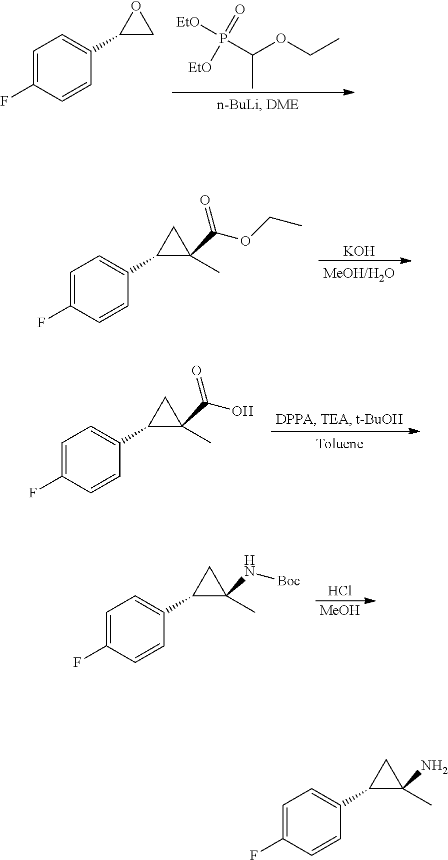

A solution of ethyl 2-(diethoxyphosphoryl)propanoate (3.45 g, 14.48 mmol, 2.00 equiv) in ethylene glycol dimethyl ether (20 mL) was treated with n-BuLi (2.5M) (5.8 mL) dropwise with stirring at 0° C. The resulting solution was stirred for 30 min at room temperature. To this was added 2-(4-fluorophenyl)oxirane (1 g, 7.24 mmol, 1.00 equiv). The resulting solution was stirred for 12 h while the temperature was maintained at 80° C. in an oil bath. The reaction mixture was cooled to RT. The reaction was then quenched by the addition of 20 mL of water. The resulting solution was extracted with ethyl acetate and the organic layers was dried and concentrated. The residue was chromatographed on silica gel and eluted with ethyl acetate/petroleum ether (1:100). This resulted in 1 g (62%) of ethyl (1R)-2-(4-fluorophenyl)-1-methylcyclopropane-1-carboxylate as yellow oil. A solution of ethyl (1R)-2-(4-fluorophenyl)-1-methylcyclopropane-1-carboxylate (1 g, 4.50 mmol, 1.00 equiv) in methanol/H₂O (10/2 mL) and potassium hydroxide (1.26 g, 22.46 mmol, 4.99 equiv) was stirred for 10 h at room temperature. The resulting solution was diluted with H₂O. The pH value of the solution was adjusted to 2 with hydrochloric acid (2 mol/L). The resulting solution was extracted with ethyl acetate and the organic layers combined and dried over anhydrous sodium sulfate and concentrated under vacuum. This resulted in 800 mg (92%) of (1R)-2-(4-fluorophenyl)-1-methylcyclopropane-1-carboxylic acid as yellow oil. A solution of (1R)-2-(4-fluorophenyl)-1-methylcyclopropane-1-carboxylic acid (400 mg, 2.06 mmol, 1.00 equiv) in toluene (10 mL) was mixed with diphenoxyphosphoryl azide (680 mg, 2.47 mmol, 1.20 equiv), and triethylamine (312 mg, 3.08 mmol, 1.50 equiv). The resulting solution was stirred for 30 min at 90° C. in an oil bath. Then, tert-butanol (2 mL) was added. The resulting solution was allowed to react, with stirring, for an additional 12 h while the temperature was maintained at 90° C. in an oil bath. The reaction mixture was cooled to room temperature and the resulting solution was diluted with ethyl acetate. The resulting mixture was washed with H₂O. The mixture was dried over anhydrous sodium sulfate and concentrated under vacuum. The residue was chromatographed on a silica gel column and eluted with ethyl acetate/petroleum ether (1:100). This resulted in 350 mg (64%) of tert-butyl N-[(1R)-2-(4-fluorophenyl)-1-methylcyclopropyl]carbamate as yellow oil. A solution of tert-butyl N-[(1R,2S)-2-(4-fluorophenyl)-1-methylcyclopropyl]carbamate (350 mg, 1.32 mmol, 1.00 equiv) in methanol (HCl) (10 mL) was stirred for 2 h at room temperature. The resulting solution was diluted with 10 mL of H₂O. The pH value of the solution was adjusted to 9 with saturated sodium bicarbonatesolution. The resulting solution was extracted with 3×10 mL of ethyl acetate and the organic layers combined and dried over anhydrous sodium sulfate and concentrated under vacuum. This resulted in 200 mg (92%) of (1R,2S)-2-(4-fluorophenyl)-1-methylcyclopropan-1-amine as yellow oil.

### Example A1: N—((S)-1-oxo-6-(((1R,2S)-2-phenylcyclopropyl)amino)-1-(pyrrolidin-1-yl)hexan-2-yl)benzamide

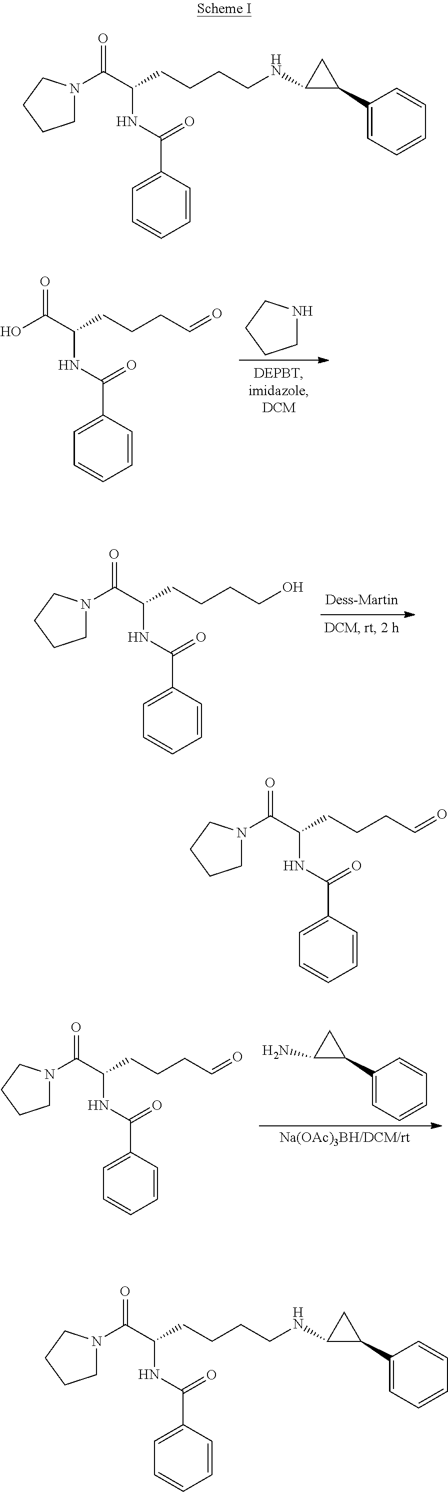

(S)-2-benzamido-6-hydroxyhexanoic acid was prepared from (S)-2-amino-6-hydroxyhexanoic acid. This material (1 g, 3.98 mmol, 1.00 equiv) in tetrahydrofuran was reacted with 3-(diethoxyphosphoryloxy)-1,2,3-benzotriazin-4(3H)-one (DEPBT) (2.4 g, 8.03 mmol, 2.00 equiv) and imidazole (542 mg, 7.97 mmol, 2.00 equiv). This was followed by the addition of a solution of pyrrolidine (283 mg, 3.98 mmol, 1.00 equiv) in tetrahydrofuran at 0° C. in 30 min. The resulting solution was stirred for 16 h at room temperature. The solution was diluted with KH₂PO₄(aq.). The aqueous layer was extracted with ethyl acetate and the organic layers were washed with brine and dried over anhydrous sodium sulfate. After filtration, solvent was removed under reduced pressure. The residue was purified by preparative HPLC and eluted with MeCN with 0.5% NH₄HCO₃. This resulted in 640 mg (53%) of (S)—N-(6-hydroxy-1-oxo-1-(pyrrolidin-1-yl)hexan-2-yl)benzamide as a light yellow oil. (S)—N-(6-hydroxy-1-oxo-1-(pyrrolidin-1-yl)hexan-2-yl)benzamide (640 mg, 2.10 mmol, 1.00 equiv) in dichloromethane (100 ml) was oxidized with Dess-Martin periodinane (DMP) (893 mg, 2.11 mmol, 1.00 equiv). The resulting solution was stirred for 30 min at 0° C. in a water/ice bath and was then diluted with Na₂SO₃(aq.) and NaHCO₃(aq.). The aqueous layers were extracted with ethyl acetate and the organic layers were washed with brine and dried over anhydrous sodium sulfate. After filtration, solvent was removed under reduced pressure. The residue was chromatographed on silica gel and eluted with ethyl acetate/petroleum ether (10:1). This gave 150 mg (24%) of (S)—N-(1,6-dioxo-1-(pyrrolidin-1-yl)hexan-2-yl)benzamide as a white solid. (S)—N-(1,6-dioxo-1-(pyrrolidin-1-yl)hexan-2-yl)benzamide (150 mg, 0.50 mmol, 1.00 equiv) was dissolved in dichloromethane (25 mL). (1R,2S)-2-phenylcyclopropanamine (66 mg, 0.50 mmol, 1.00 equiv) was added. After stirring 5 minutes, sodium triacetoxyborohydride (252 mg, 1.19 mmol, 2.40 equiv) was added. The resulting solution was stirred for 30 min at 0° C. After the reaction was completed, the resulting solution was diluted with sat.NaHCO₃. Then it was extracted with dichloromethane. The organic layers were washed with brine and dried over anhydrous sodium sulfate. Solvent was removed under reduced pressure and the residue was purified by Prep-HPLC (CAN/H₂O with 0.5% NH₄HCO₃). This resulted in 29 mg (14%) of N—((S)-1-oxo-6-(((1R,2S)-2-phenylcyclopropyl)amino)-1-(pyrrolidin-1-yl)hexan-2-yl)benzamide as colorless oil. ¹H NMR (300 MHz, CD₃OD-d₄) δ ppm: 7.85 (d, J=7.5 Hz, 2H), 7.60-7.00 (m, 8H), 4.85-4.75 (m, 1H), 3.92-3.80 (m, 1H), 3.70-3.30 (m, 4H), 2.74 (t, J=7.2 Hz, 1H), 2.36-2.28 (m, 1H), 2.07-1.75 (m, 7H), 1.74-1.37 (m, 4H), 1.10-0.95 (m, 2H); MS (ES, m/z): 420 (M+H).

### Example A2: N—((S)-1-oxo-6-(((1R,2S)-2-phenylcyclopropyl)amino)-1-(piperidin-1-yl)hexan-2-yl)benzamide

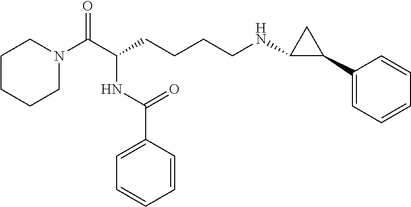 [SMILES: O=C(N[C@@H](CCCCN[C@@H]1C[C@H]1c1ccccc1)C(=O)N1CCCCC1)c1ccccc1]

N—((S)-1-oxo-6-(((1R,2S)-2-phenylcyclopropyl)amino)-1-(piperidin-1-yl)hexan-2-yl)benzamide was prepared in the same manner as was described for the synthesis of N—((S)-1-oxo-6-(((1R,2S)-2-phenylcyclopropyl)amino)-1-(pyrrolidin-1-yl)hexan-2-yl)benzamide. (S)-2-benzamido-6-hydroxyhexanoic acid was coupled with piperidine using 3-(diethoxyphosphoryloxy)-1,2,3-benzotriazin-4(3H)-one and imidazole. The resultant alcohol (S)—N-(6-hydroxy-1-oxo-1-(piperidin-1-yl)hexan-2-yl)benzamide was oxidized under Dess-Martin conditions to the aldehyde (S)—N-(1,6-dioxo-1-(piperidin-1-yl)hexan-2-yl)benzamide. This was coupled with (1R,2S)-2-phenylcyclopropanamine under reductive amination conditions (Na(OAc)₃BH) to yield the desired product N—((S)-1-oxo-6-(((1R,2S)-2-phenylcyclopropyl)amino)-1-(piperidin-1-yl)hexan-2-yl)benzamide as a colorless oil. ES, m/z=434 (M+H). ¹H NMR (300 MHz, CD₃OD-d₄) δ ppm: 7.86 (d, J=7.2 Hz, 2H), 7.70-7.40 (m, 3H), 7.30-7.15 (m, 2H), 7.15-7.08 (m, 1H), 7.06 (d, J=7.2 Hz, 2H), 5.15-5.00 (m, 1H), 3.80-3.60 (m, 2H), 3.60-3.40 (m, 2H), 2.34 (t, J=7.2 Hz, 2H), 2.40-2.30 (m, 1H), 2.10-1.40 (m, 4H), 1.15-1.00 (m, 2H).

### Example A3: 4-fluoro-N—((S)-6-(((1R,2S)-2-(4-fluorophenyl)cyclopropyl)amino)-1-(4-methylpiperazin-1-yl)-1-oxohexan-2-yl)benzamide

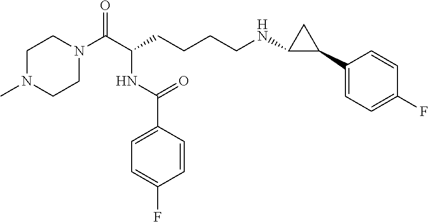 [SMILES: CN1CCN(C(=O)[C@H](CCCCN[C@@H]2C[C@H]2c2ccc(F)cc2)NC(=O)c2ccc(F)cc2)CC1]

4-fluoro-N—((S)-6-(((1R,2S)-2-(4-fluorophenyl)cyclopropyl)amino)-1-(4-methylpiperazin-1-yl)-1-oxohexan-2-yl)benzamide was prepared in a manner analogous to Example A2. The alcohol 4-fluoro-N—((S)-6-(((1R,2S)-2-(4-fluorophenyl)cyclopropyl)amino)-1-(4-methylpiperazin-1-yl)-1-oxohexan-2-yl)benzamide was prepared by reduction of (S)-2-(4-fluorobenzamido)hexanedioic acid with Me₂S—BH₃. This type of reduction was used to prepare similar alcohols (e.g. The alcohol starting material (S)-2-benzamido-6-hydroxyhexanoic acid for the synthesis of N—((S)-1-oxo-6-(((1R,2S)-2-phenylcyclopropyl)amino)-1-(pyrrolidin-1-yl)hexan-2-yl)benzamide (Example A1)). Into a 1000-mL 3-necked round-bottom flask purged and maintained with an inert atmosphere of nitrogen, was placed a solution of (S)-2-(4-fluorobenzamido)hexanedioic acid (10 g, 35.30 mmol, 1.00 equiv) in tetrahydrofuran (300 ml). Then a solution of Me₂S—BH₃ (11 mL, 3.00 equiv) in tetrahydrofuran (50 ml) was added at 0° C. The resulting solution was stirred for 3 h at 0° C. in an ice/salt bath. The reaction was then quenched by the addition of 20 ml of methanol. The resulting mixture was concentrated under vacuum. The resulting solution was diluted with 300 ml of sat.Na₂CO₃. The resulting solution was extracted with 3×100 mL of ethyl acetate and the aqueous layers combined. The pH value of the solution was adjusted to 2 with hydrochloric acid (2 mol/L). The resulting solution was extracted with 3×200 ML of ethyl acetate and the organic layers combined. The resulting mixture was washed with 1×500 mL of brine. The mixture was dried over anhydrous sodium sulfate. The solids were filtered out. The resulting mixture was concentrated under vacuum. This resulted in 6 g (63%) of (S)-2-(4-fluorobenzamido)-6-hydroxyhexanoic acid as colorless oil. This material was reacted with N-methyl piperazine followed by Dess-Martin oxidation and coupling via reductive amination with (1R,2S)-2-(4-fluorophenyl)cyclopropanamine in the manner described for the synthesis of N—((S)-1-oxo-6-(((1R,2S)-2-phenylcyclopropyl)amino)-1-(pyrrolidin-1-yl)hexan-2-yl)benzamide (Example A1) to yield the desired product 4-fluoro-N—((S)-6-(((1R,2S)-2-(4-fluorophenyl)cyclopropyl)amino)-1-(4-methylpiperazin-1-yl)-1-oxohexan-2-yl)benzamide as colorless oil. ES, m/s=485*M+H). ¹H NMR (300 MHz, CD₃OD-d₄) δ ppm: 7.83 (dd, J₁=5.4 Hz, J₂=1.4 Hz, 2H), 7.18-7.04 (m, 3H), 7.00-6.87 (m, 4H), 5.17-5.05 (m, 1H), 3.78-3.50 (m, 4H), 2.71 (t, J=6.9 Hz, 2H), 2.30 (s, 3H), 2.28-2.21 (m, 1H), 1.90-1.78 (m, 2H), 1.72-1.31 (m, 9H), 1.07-0.96 (m, 1H), 0.94-0.86 (m, 1H).

### Example 158: N-[(2S)-1-(4-(methyl)piperazin-1-yl)-5-[[(1R,2S)-2-(4-fluorophenyl)-cyclopropyl]amino]-1-oxopentan-2-yl]-4-(1H-1,2,3-triazol-1-yl)benzamide (Compound 2; Free Base of Compound 1)

N-[(2S)-1-(4-(methyl)piperazin-1-yl)-5-[[(1R,2S)-2-(4-fluorophenyl)-cyclopropyl]amino]-1-oxopentan-2-yl]-4-(1H-1,2,3-triazol-1-yl)benzamide (Compound 2) was prepared according to the method of Scheme II.

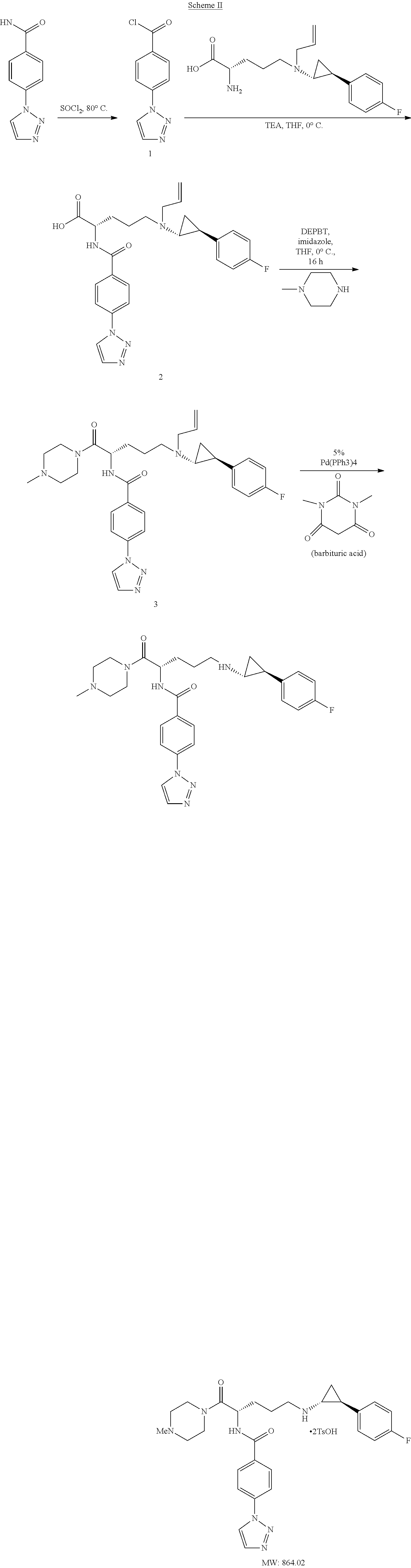

4-(1H-1,2,3-triazolyl-1-yl)benzoyl chloride (1) In a 100-mL round-bottom flask were combined 4-(1H-1,2,3-triazol-1-yl)benzoic acid (1 g, 5.29 mmol, 1.00 equiv) and thionyl chloride (20 mL). The resulting solution was stirred for 16 h at 80° C. in an oil bath. The resulting mixture was then concentrated under reduced pressure, affording 1 g (91%) of intermediate (1) as a yellow solid.

(2S)-5-[[(1R,2S)-2-(4-fluorophenyl)cyclopropyl](propen-3-yl)amino]-2-[[4-(1H-1,2,3-triazol-1-yl)phenyl]formamido]pentanoic acid (2) In a 100-mL round-bottom flask were combined (2S)-2-amino-5-[(1R,2S)-2-(4-fluorophenyl)cyclopropyl](prop-2-en-1-yl)aminopentanoic acid (500 mg, 1.63 mmol, 1.00 equiv), Et₃N (494 mg, 4.88 mmol, 3.00 equiv) and THF (20 mL). This was followed by the addition of a solution of intermediate (1) from the previous step (1 g, 4.82 mmol, 2.95 equiv) in THF (20 mL) dropwise with stirring at 0° C. in 30 min. The resulting solution was stirred for 1 h at 0° C. in an ice/salt bath, then concentrated under reduced pressure, and applied onto a silica gel column with CH₂Cl₂/methanol (10:1). The collected fractions were combined and concentrated under reduced pressure, affording 400 mg (51%) of intermediate (2) as a off-white solid.

N-[(2S)-1-(4-(methyl)piperazin-1-yl)-5-[[(1R,2S)-2-(4-fluorophenyl)-cyclopropyl](prop-2-en-1-yl)amino]-1-oxopentan-2-yl]-4-(1H-1,2,3-triazol-1-yl)benzamide (3) In a 100-mL round-bottom flask were combined intermediate (2) from the previous step (400 mg, 0.84 mmol, 1.00 equiv), DEPBT (375 mg, 1.25 mmol, 1.50 equiv), and THF (20 mL), followed by the addition of imidazole (85 mg, 1.25 mmol, 1.50 equiv). The mixture was stirred for 30 min at 0° C., at which point 1-methylpiperazine (127 mg, 1.27 mmol, 1.50 equiv) was added dropwise with stirring at 0° C. in 3 min. The resulting solution was stirred for 16 h at 20° C., then concentrated under reduced pressure. The residue was applied onto a silica gel column with CH₂Cl₂/methanol (10:1). The collected fractions were combined and concentrated under vacuum, affordin 300 mg (64%) of intermediate (3) as a yellow solid.

N-[(2S)-1-(4-(methyl)piperazin-1-yl)-5-[[(1R,2S)-2-(4-fluorophenyl)-cyclopropyl]amino]-1-oxopentan-2-yl]-4-(1H-1,2,3-triazol-1-yl)benzamide (Example 158; Compound 2) Into a 100-mL round-bottom flask purged and maintained with an inert atmosphere of nitrogen, was placed N-[(2S)-5-[[(1R,2S)-2-(4-fluorophenyl)cyclopropyl](prop-2-en-1-yl)amino]-1-(4-methylpiperazin-1-yl)-1-oxopentan-2-yl]-4-(1H-1,2,3-triazol-1-yl)benzamide (300 mg, 0.54 mmol, 1.00 equiv), 1,3-dimethyl-1,3-diazinane-2,4,6-trione (210 mg, 1.34 mmol, 2.50 equiv), Pd(PPh₃)₄ (155 mg, 0.13 mmol, 0.25 equiv). The resulting solution was stirred for 2 h at 45° C. in an oil bath. The resulting mixture was concentrated under vacuum. The crude product (10 mL) was purified by Flash-Prep-HPLC. This resulted in 65 mg (23%) of Example 158 as a yellow solid.

Alternatively, Example 158 and its bis-tosylate salt (Compound 2 bis-tosylate salt, “Compound 1”)

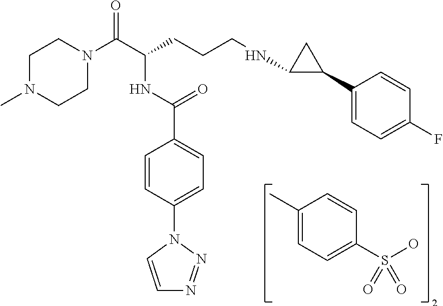

may be prepared by the method of Scheme III:

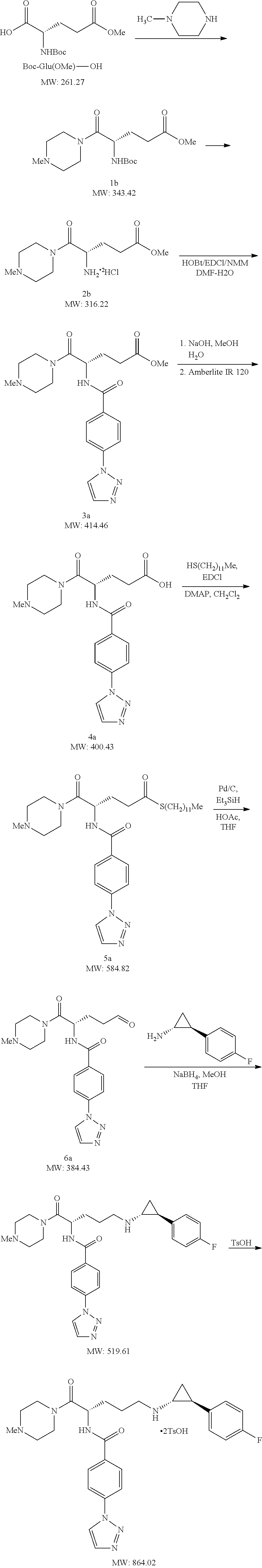

Compounds disclosed herein, including Compound 1, may also be synthesized as disclosed in US20160237043, WO2018035259 and WO2018035249.

The compounds herein may be synthesized using methods analogous to those described herein and known in the art, using appropriate starting materials and reagents. In the following structures, it should be understood that mixtures of or single isomers, such as racemic mixtures and alternate enantiomers, zwitterions, and the like may be prepared, e.g. by using appropriate L- or D-isomer, or chiral or achiral compound, as a staring material or reagent, or by employing a separation step.

Therefore, in certain embodiments in the compounds below, the configuration of the substituents off the cyclopropylamine is trans to the phenyl. In certain embodiments, the trans configuration is R, S; in others, it is S, R.

In certain embodiments, the compound is:

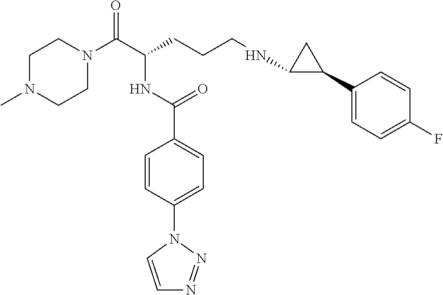 [SMILES: CN1CCN(C(=O)[C@H](CCCN[C@@H]2C[C@H]2c2ccc(F)cc2)NC(=O)c2ccc(-n3ccnn3)cc2)CC1]

(“Compound 2”) or a salt, polymorph, or solvate thereof.

In certain embodiments, the compound is a salt of the formula:

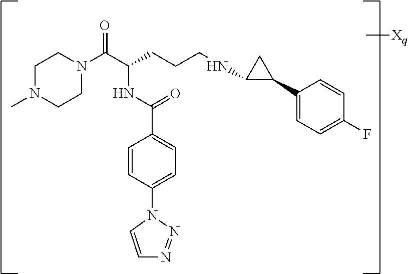

or a polymorph or solvate thereof, wherein:
X is chosen from tosylate, sulfate, tartrate, oxalate, besylate, fumarate, citric, esylate, and malate; and
   q is an integer chosen from 1 and 2.

In certain embodiments, X is tosylate.

In certain embodiments, q is 2.

In certain embodiments, the compound is

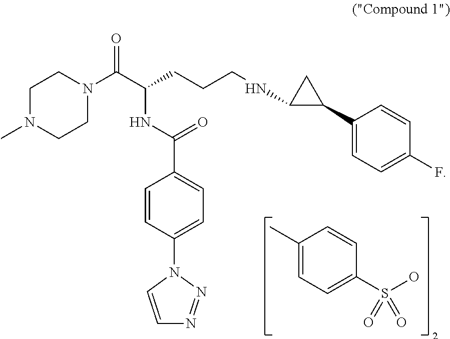

The compounds disclosed above, or any subset or species of them, may be used in any of the methods of treatment and effecting of clinically/therapeutically relevant endpoints described herein.

In certain embodiments, a compound as disclosed herein is provided for use as a medicament.

In certain embodiments, a compound as disclosed herein is provided for use in the manufacture of a medicament for the prevention or treatment of a disease or condition, or effecting of a clinically relevant endpoint, as discussed herein.

In certain embodiments, a pharmaceutical composition is provided which comprises a compound as disclosed herein, together with a pharmaceutically acceptable carrier.

In certain embodiments, the pharmaceutical composition is formulated for oral administration.

In certain embodiments, the pharmaceutical composition additionally comprises another therapeutic agent.

### Methods of Treatment of Disease and Uses in Medicaments

Provided herein are methods for treating or preventing a myeloproliferative neoplasm, the method comprising administering to a subject in need thereof an LSD1 inhibitor compound as disclosed herein.

In certain embodiments, the method effects or results in one or more of the following:
suppression of proliferation of malignant myeloid cells in a subject in need thereof;
reduction of the concentration of one or more protein growth factors (e.g., platelet-derived growth factor, vascular endothelial growth factor, transforming growth factor beta 1 or platelet factor 4 (aka CXCL4)) secreted by bone marrow cells (e.g., megakaryocytes) that activate one or more cell types that secrete reticulin and collagen (e.g. stromal cells, bone marrow-resident fibroblasts, or myofibroblasts) in a subject in need thereof;
reduction of the concentration of one or more protein growth factors (e.g., platelet-derived growth factor, vascular endothelial growth factor, transforming growth factor beta 1 or platelet factor 4 (aka CXCL4)) secreted by bone marrow cells (e.g., megakaryocytes) that impair the function of bone marrow osteoclasts to reduce the amount of bone marrow osteosclerosis in a subject in need thereof;
reduction of reticulin and/or collagen bone marrow fibrosis in a subject in need thereof;
reduction of plasma levels of one or more inflammatory cytokines in a subject in need thereof;
reduction of the malignant cell burden measured by the mutant allele frequency of myeloid cells in a subject in need thereof;
elimination of malignant myeloid cells in a subject in need thereof;
reduction of a pathologically elevated red blood cell mass in a subject in need thereof;
reduction of the mass of malignant myeloid cells in a subject in need thereof;
reduction of abnormal spleen size or volume in a subject in need thereof;
reduction of the amount of extramedullary hematopoiesis in a subject in need thereof;
improvement of the quality of life (QOL) measured by validated patient-reported QOL assessments in a subject in need thereof;
reduction of the constitutional symptoms of myelofibrosis measured by patient-reported surveys in a subject in need thereof;
extension of the life span of a subject with myelofibrosis in need thereof;
delay or prevention of the progression of myelofibrosis to acute myeloid leukemia in a subject in need thereof;
reduction of platelet counts in a subject in need thereof;
reduction of a pathologically elevated red blood cell mass in a subject in need thereof;
reduction of elevated an elevated level of bone marrow cells of granulocytic lineage in a subject in a subject in need thereof;
reduction of bone marrow cellularity to age-adjusted normocellularity with fewer than 5% blast cells in a subject in need thereof;
maintenance of the bone marrow blast count or reducing the bone marrow blast count to <5% in a subject in need thereof
reduction of the frequency of thrombosis and hemorrhage in a subject in need thereof;
reduction of the frequency of transfusions of red blood cells in a subject in need thereof;
increases hemoglobin to a value >100 g/L and less than the upper limit of age-and sex adjusted normal in a MF patient;
reducing the hematocrit in a male patient with PV to <45% or reducing the hematocrit in a female patient with PV to ≤42%;
reduces hemoglobin level in a PV patient to <160 g/L in a PV patient; and/or
decreases red cell mass in a PV patient to ≤5.2M/mL.

In certain embodiments, the method effects or results in two or more of the foregoing. In certain embodiments, the method effects or results in three or more of the foregoing. In certain embodiments, the method effects or results in two or more of the foregoing other than reduces platelet counts in a subject in need thereof. In certain embodiments, the one, two, three, or more of the foregoing is limited by a recitation below.

In certain embodiments, the subject in need is one who has a myeloproliferative neoplasm. In certain embodiments, the myeloproliferative neoplasm is chosen from polycythemia vera (PV), essential thrombocythemia (ET), myelofibrosis (MF), chronic myelogenous leukemia (CML), chronic neutrophilic leukemia (CNL), and chronic eosinophilic leukemia (CEL). In certain embodiments, the myeloproliferative neoplasm is chosen from polycythemia vera (PV), essential thrombocythemia (ET), and myelofibrosis (MF). In certain embodiments, the myeloproliferative neoplasm is myelofibrosis. In certain embodiments, the myelofibrosis is chosen from primary myelofibrosis (PMF) and post PV/ET myelofibrosis. In certain embodiments, the myeloproliferative neoplasm is primary myelofibrosis (PMF). In certain embodiments, the myeloproliferative neoplasm is post PV/ET myelofibrosis. In certain embodiments, the myeloproliferative neoplasm is essential thrombocythemia. In certain embodiments, the myeloproliferative neoplasm is polycythemia vera. In certain embodiments, the myeloproliferative neoplasm is chronic myelogenous leukemia. In certain embodiments, the myeloproliferative neoplasm is chronic neutrophilic leukemia. In certain embodiments, the myeloproliferative neoplasm is chronic eosinophilic leukemia. In certain embodiments, the patient is a human.

Provided herein is a method for suppressing proliferation of malignant myeloid cells, in a subject in need thereof, the method comprising administering a therapeutically effective and non-deleterious amount of an LSD1 inhibitor. In certain embodiments, the malignant myeloid cells have mutations in one or more genes chosen from Janus Kinase 2 (JAK2), myeloproliferative leukemia virus oncogene (MPL) and calreticulin (CALR). In certain embodiments, the method further comprises the step of determining whether said subject has mutations in one or more genes chosen from Janus Kinase 2 (JAK2), myeloproliferative leukemia virus oncogene (MPL) and calreticulin (CALR). In certain embodiments, the malignant myeloid cells are malignant stem cells. In certain embodiments, reduction of the malignant myeloid cells is measured by the frequency of the mutant allele burden as measured by PCR or sequencing or other methods known in the art. In certain embodiments, the malignant myeloid cells are reduced by at least 50%. In certain embodiments, the malignant myeloid cells are reduced by 2 or more logs (100× or more).

Provided herein is a method for reducing reticulin and/or collagen bone marrow fibrosis in a subject in need thereof, the method comprising administering a therapeutically effective and non-deleterious amount of an LSD1 inhibitor. In certain embodiments, the bone marrow fibrosis is reticulin bone marrow fibrosis. In certain embodiments, the bone marrow fibrosis is collagen bone marrow fibrosis. In certain embodiments, the bone marrow fibrosis is reticulin and collagen bone marrow fibrosis. In certain embodiments, the reticulin and/or collagen bone marrow fibrosis is reduced by at least one grade, e.g., from 3 to 2, or from 2 to 1, or from 1 to 0. In certain embodiments, the reticulin and/or collagen bone marrow fibrosis is reduced by at least two grades.

In certain embodiments, the subject has mutations in one or more genes chosen from Janus Kinase 2 (JAK2), myeloproliferative leukemia virus oncogene (MPL) and calreticulin (CALR). In certain embodiments, the LSD1 inhibitor is an LSD1 inhibitor compound as disclosed herein. The mutations may be assessed by methods known in the art, for example those disclosed in Spivak J, “Narrative Review: Thrombocytosis, polycythemia vera, and JAK2 mutations: the phenotypic mimicry of chronic myeloproliferation,” Annals of Internal Medicine 2010 152(5):300-306 or Zhan H and Spivak J L, “The diagnosis and management of polycythemia vera, essential thrombocythemia, and primary myelofibrosis in the JAK2 V617F era,” Clin Adv Hematol Oncol, 2009 May; 7(5):334-42.

Provided herein is a method for reducing plasma levels of one or more inflammatory cytokines in a subject in need thereof, the method comprising administering a therapeutically effective and non-deleterious amount of an LSD1 inhibitor. In certain embodiments, one or more of the inflammatory cytokines is chosen from interferon gamma (IFNγ), tumor necrosis factor alpha (TNFα), interleukin 1β (IL-1β), interleukin 6 (IL-6), interleukin 8 (IL-8), interleukin 10 (IL-10), interleukin 12 (IL-12), interleukin 15 (IL-15), interleukin 17 (IL-17), CXCL4 (PF4), and CXCL10 (IP10).

In certain embodiments, the measured cytokine or cytokines are reduced to about the following levels, or below:

IL-6 is reduced to below about 9 pg/mL;

IL-8 is reduced to below about 18 pg/mL;

IL-10 is reduced to below about 51 pg/mL;

IL-12 is reduced to below about 182 pg/mL;

IL-15 is reduced to below about 38 pg/mL;

TNFα is reduced to below about 15 pg/mL; and/or

INFγ is reduced to below about 23 pg/mL.

In certain embodiments, two, three, four, five, or more of the inflammatory cytokines are reduced.

Provided herein is a method for reducing the mass of malignant myeloid cells in a subject in need thereof, the method comprising administering a therapeutically effective and non-deleterious amount of an LSD1 inhibitor. In certain embodiments, the mass of malignant myeloid cells is measured by flow cytometry immunophenotyping. In certain embodiments, the mass of malignant myeloid cells is measured by the frequency of the mutant allele, a ratio of the number of cells with the causative MPN mutations (MPL, CALR or JAK2) over the total number of cells that contain both the wild-type and mutant alleles.

Provided herein is a method for reducing mutant allele burden in a subject in need thereof, the method comprising a therapeutically effective amount of an LSD1 inhibitor. In certain embodiments, the mutant allele is an allele of one or more genes chosen from Janus Kinase 2 (JAK2), myeloproliferative leukemia virus oncogene (MPL) and calreticulin (CALR). In certain embodiments, the LSD1 inhibitor is an LSD1 inhibitor compound as disclosed herein. In certain embodiments, the mutant allele burden is reduced by about 50% of a subject's (or the subject pool's average) mutant allele burden of mutated Janus Kinase 2 (JAK2), myeloproliferative leukemia virus oncogene (MPL) or calreticulin (CALR). In certain embodiments, the reduction in mutant allele burden is measured within patient(s) after treatment and compared to the level prior to treatment to the level after a course of treatment. In certain embodiments, the mutant allele burden is reduced to a level where mutant alleles of Janus Kinase 2 (JAK2), myeloproliferative leukemia virus oncogene (MPL) and calreticulin (CALR) are undetectable. Mutant allele burden may be assessed by methods known in the art, including those disclosed above.

Provided herein is a method for reducing a pathologically elevated red blood cell mass in a subject in need thereof, the method comprising administering a therapeutically effective and non-deleterious amount of an LSD1 inhibitor. In certain embodiments, the subject has polycythemia vera. In certain embodiments, the subject has a mutation in Janus Kinase 2 (JAK2). In certain embodiments, the elevated red blood cell mass is inferred by the measure of the hematocrit or blood hemoglobin. In certain embodiments, measured the hematocrit or the hemoglobin should be reduced to the normal range appropriate to gender. For example, in certain embodiments:
blood hemoglobin will be reduced to less than 16.5 g/dL for a male PV patient or to less than 16.0 g/dL for a female PV patient;
hematocrit will be reduced to less than 49% for a male PV patient or to less than 48% for a female PV patient.
   In certain embodiments, the elevated red blood cell mass is measured by isotopic red cell mass measurement. In certain embodiments the increased red cell mass is greater than 25% above mean normal predicted value.

Provided herein is a method for reducing an elevated white blood cell count in a subject in need thereof, the method comprising administering a therapeutically effective and non-deleterious amount of an LSD1 inhibitor. In certain embodiments, subject has chronic neutrophilic leukemia.

Also provided herein is a method for reducing an elevated level of bone marrow cells of granulocytic lineage in a subject in need thereof, the method comprising administering a therapeutically effective and non-deleterious amount of an LSD1 inhibitor. In certain embodiments, the bone marrow cells of granulocytic lineage are reduced to a value within the normal range. Also provided herein is a method for, in a subject in need thereof, reducing bone marrow cellularity to age-adjusted normocellularity with fewer than 5% blast cells, the method comprising administering a therapeutically effective amount of an LSD1 inhibitor. In certain embodiments, subject has chronic neutrophilic leukemia.

Provided herein is a method for increasing hemoglobin to >100 g/L up to a level less than the upper limit of age-and sex adjusted normal in a subject in need thereof, the method comprising administering a therapeutically effective and non-deleterious amount of an LSD1 inhibitor.

Also provided is a method for a) reducing hemoglobin level in a PV patient to <160 g/L, or b) decreasing red cell mass in a PV patient, wherein the decrease is inferred from hemoglobin levels Hb of <160 g/L, either comprising administering a therapeutically effective and non-deleterious amount of an LSD1 inhibitor. Also provided is a method for increasing hemoglobin to >100 g/L in a MF patient, comprising administering a therapeutically effective amount of an LSD1 inhibitor. Also provided is a method for increasing hemoglobin to a value >100 g/L and less than the upper limit of age-and sex adjusted normal in a MF patient, the method comprising administering a therapeutically effective amount of an LSD1 inhibitor. In certain embodiments, said subject has a mutation in one or more genes chosen from Janus Kinase 2 (JAK2), myeloproliferative leukemia virus oncogene (MPL) and calreticulin (CALR). In certain embodiments, said subject has essential thrombocythemia. In certain embodiments, the transfusion burden of said patient is reduced.

Provided herein is a method for reducing abnormal spleen size or volume in a subject in need thereof, the method comprising administering a therapeutically effective and non-deleterious amount of an LSD1 inhibitor. In certain embodiments, said subject has a mutation in one or more genes chosen from Janus Kinase 2 (JAK2), myeloproliferative leukemia virus oncogene (MPL) and calreticulin (CALR).

Provided herein is a method for reducing the amount of extramedullary hematopoiesis in a subject in need thereof, the method comprising administering a therapeutically effective and non-deleterious amount of an LSD1 inhibitor. In certain embodiments, said subject has a mutation in one or more genes chosen from Janus Kinase 2 (JAK2), myeloproliferative leukemia virus oncogene (MPL) and calreticulin (CALR). In certain embodiments, the amount of extramedullary hematopoiesis is measured by splenomegaly. In certain embodiments, splenomegaly in said subject is reduced by at least about 30%, at least about 35%, at least about 40%, or least about 45%. In certain embodiments, splenomegaly in said subject is reduced by at least 35%. In certain embodiments, splenomegaly in is reduced by at least 35% in about 50% of patients.

Provided herein is a method for reducing the constitutional symptoms of myelofibrosis, as measured by patient-reported surveys in a subject in need thereof, the method comprising administering a therapeutically effective and non-deleterious amount of an LSD1 inhibitor. In certain embodiments, said constitutional symptoms comprise one or more symptoms chosen from fatigue, early satiety, abdominal discomfort, inactivity, problems with concentration, numbness and/or tingling in the hands and feet, night sweats, pruritis, bone pain, fever greater than 100° F., and unintentional weight loss.

In certain embodiments, said patient-reported survey is the Myeloproliferative Neoplasm Symptom Assessment Form (MPN-SAF). The MPN-SAF is a validated clinical assessment form for the most common symptoms of myeloproliferative neoplasms, in which patients self-reports their score, on a scale of 1-10, of various common symptoms, where 1 is the most favorable or the symptom is absent, and 10 is the least favorable or the symptom is the worst imaginable. See, e.g., Scherber R et al., The Myeloproliferative Neoplasm Symptom Assessment Form (MPN-SAF): International Prospective Validation and Reliability Trial in 402 patients,” Blood 118(2):401-08 (2014). Either the full or abbreviated forms may be administered to the patient. In the abbreviated version, a “total symptom score” (TSS) may be calculated from the ten most clinically relevant symptoms from the 17-item MPN-SAF: worst fatigue, concentration, early satiety, inactivity, night sweats, itching, bone pain, abdominal discomfort, weight loss, and fever. The MPN-SAF TSS thus has a possible range of 0 to 100. Quality of life scores are defined as “clinically deficient” when they rate as at least 4 of 10; “moderate” if symptoms are rated as ≥4 of 10 or ≤6 of 10; and “severe” if symptoms are rated as ≥7 of 10. For patients who complete at least six of these 10 items on the BFI and MPN-SAF, the MPN TSS is computed as the average of the observed items multiplied by 10 to achieve a 0-to-100 scale. See, e.g., Emanuel R M et al., “Myeloproliferative neoplasm (MPN) symptom assessment form total symptom score: prospective international assessment of an abbreviated symptom burden scoring system among patients with MPNs,” J Clin Oncol 30(33):4098-103 (2012).

In certain embodiments, the total symptom score (MPN-SAF:TSS) is reduced by at least 50%.

In certain embodiments, said patient-reported survey is the Myelofibrosis Symptom Assessment Form (MF-SAF). See, e.g., Mesa R A et al., “The Myelofibrosis Symptom Assessment Form (MFSAF): an evidence-based brief inventory to measure quality of life and symptomatic response to treatment in myelofibrosis,” Leuk Res. 33(9):1199-203 (2009). In certain embodiments, the MF-SAF total symptom score is reduced by at least 50%.

In certain embodiments:
the subject has a mutation in one or more genes chosen from Janus Kinase 2 (JAK2), myeloproliferative leukemia virus oncogene (MPL) and calreticulin (CALR);
the subject has a myeloproliferative neoplasm;
the subject has a myeloproliferative neoplasm chosen from polycythemia vera (PV), essential thrombocythemia (ET), and myelofibrosis;
the subject has myelofibrosis;
the subject has myelofibrosis chosen from primary myelofibrosis (PMF) and post PV/ET myelofibrosis;
the subject has post PV/ET myelofibrosis (MF);
the subject has primary myelofibrosis (PMF);
the subject has polycythemia vera;
the subject has essential thrombocythemia;
the subject has chronic myelogenous leukemia;
the subject has chronic neutrophilic leukemia; or
the subject has chronic eosinophilic leukemia;
the subject is a human; and/or
the LSD1 inhibitor is an LSD1 inhibitor compound as disclosed herein.

Also provided are embodiments wherein any method embodiment above may be combined with any one or more of these embodiments, provided the combination is not mutually exclusive. As used herein, two embodiments are “mutually exclusive” when one is defined to be something which cannot overlap with the other. For example, an embodiment wherein the disorder to be treated is primary myelofibrosis (PMF) is mutually exclusive with an embodiment wherein the disorder to be treated is post PV/ET myelofibrosis (MF), because these classifications are the product of different diagnoses. However, an embodiment wherein the disorder to be treated is PMF is not mutually exclusive with an embodiment wherein reticulin and/or collagen bone marrow fibrosis is reduced, because reticulin and/or collagen bone marrow fibrosis occur in PMF.

The methods disclosed above, or any subset or species of them, may use any of the compounds disclosed above as LSD1 inhibitors, either a discrete chemical species or as described by one of the formulae or embodiments, or a pharmaceutical composition comprising them.

### EXAMPLES

Presented below are biological assays and clinical trials demonstrating the utility of the compositions and methods disclosed herein.

### Biological Activity

Compounds disclosed herein have been shown to be inhibitors of LSD1, as disclosed for example in WO 2015/021128 and WO 2016/130952, or any of the references cited above, the contents of which are hereby incorporated by reference.

### Example 1: Phase 1/2A and Phase 2B Clinical Trials in Myelofibrosis

A multi-center, open-label study evaluating the safety, tolerability, steady-state pharmacokinetics and pharmacodynamics of Compound 1 administered orally once daily in patients with high-risk MF, including primary myelofibrosis (PMF), post-polycythemia vera myelofibrosis (PPV-MF), and post-essential thrombocythemia myelofibrosis (PET-MF) (collectively referred to as ‘MF’) was initiated as a Phase 1/2A study, and was expanded to a Phase 2b study.

The Phase 1/2A portion of the study assessed: the safety of the original starting dose, 0.25 mg/kg/d; an 85-day duration of treatment with a subsequent washout period of up to 28 days; and, pharmacokinetic and drug concentration measurements. Patients demonstrating clinical benefit could resume treatment for additional 12 week cycles. With transition to a Phase 2b study, changes supported by the earlier pharmacokinetic and pharmacodynamic studies and safety assessments were implemented, including: an increased starting dose of 0.5 mg/kg/d with larger titration increments; a 168-day (24 week) duration of treatment, with continuous dosing via removal of the washout period; elimination of PK and drug concentration sampling; and, a reduced visit schedule.

This study was conducted at multiple sites. Up to 50 patients, eighteen years of age or older, with high-risk myelofibrosis were treated. The primary objectives included safety and tolerability, pharmacokinetics (PK; Phase 1/2A only) and spleen volume reduction (SVR). Exploratory endpoints included improvement in constitutional symptoms demonstrated by reduction in total symptoms scores (TSS) derived from the MPN-SAF in Phase 1/2A and using the MPN-SAF TSS instrument in Phase 2B, cytokines, and bone marrow (BM) fibrosis. Key inclusion criteria included: high- or intermediate-2 risk myelofibrosis; per the Investigator's judgment have failed (refractory or resistant to, inadequately controlled by or intolerant of), or are not a candidate for, available approved therapy including ruxolitinib; platelet count ≥100K/μL; and, circulating blasts ≤10%.

Dosing was tailored using platelet count as a biomarker for the effect of bomedemstat activity on megakaryocyte function and activity. The megakaryocytes, the cell in bone marrow and elsewhere that make platelets, are central to the pathogenesis of myelofibrosis and essential thrombocythemia. In both conditions, somatic mutations in bone marrow stem cells result in mature megakaryocytes that produce excess platelets and biologically active proteins that alter the bone marrow niche as well as spill into the circulation resulting in symptoms characteristic of these conditions such as itching and fatigue.

One strategy to reduce the excess products of megakaryocytes is to target megakaryocyte maturation and function. The effectiveness of a treatment targeting the megakaryocyte may be quantified by measuring the products of megakaryocytes, e.g., platelets in circulation or inflammatory cytokines and growth factors in plasma or serum.

Dosing of such a treatment can be made more precise by titrating the dose to lower the platelet count to a specific range.

In the Phase 1/2a portion of the study, patients started at the presumed sub-therapeutic dose of 0.25 mg/kg/d. Dose-adjustments were made weekly (the lifespan of a human platelet) with dose-titration, either upward or downward, contingent on platelet values at the time of evaluation. Upward titrations were made in increments of 0.125 or 0.0625 mg/kg/d as shown below. Downward titrations were made in decrements of 50% of the current dose. The calculated effective dose was anticipated to be ˜1 mg/kg QD though this did not represent an upper limit; the dose needed to achieve the optimal therapeutic effect was expected to vary among patients and possibly change over time. The titration target platelet count expected to be associated with most efficacious therapeutic effect was ≥50,000 to <100,000 μL (50-100×10⁹/L). The Phase 1/2a titration and re-challenge rules based on weekly evaluation of platelet counts are noted below in Table 1.

TABLE 1
Titration and Re-challenge Rules for
the Phase 1/2a Portion of the Study

| Platelet (Plt Assessment) | | Titration and Re-challenge Rules | | |
|---|---|---|---|---|
| Plt Count | % Plt | | Titration | Re-challenge |
| (×10⁹/L) | Reduction | Titration?* | Rule* | Rule |
| ≥100 | <50% from | Up-titrate | Add 0.125 | N/A |
| | previous week | | mg/kg/d | |
| ≥100 | >50% from | Up-titrate | Add 0.0625 | N/A |
| | previous week | | mg/kg/d | |
| 75-99 | <30% from | Up-titrate | Add 0.0625 | N/A |
| | previous week | | mg/kg/d | |
| 75-99 | >30% from | Maintain | N/A | N/A |
| | previous week | current dose | | |
| 50-74 | N/A | Maintain | N/A | N/A |
| | | current dose | | |
| 25-49 | N/A | Down-titrate | 50% of | N/A |
| | | | current dose | |
| <25 | N/A | HOLD | N/A | At 50% of |
| | | DOSE | | previous dose |
| | | | | when platelets |
| | | | | return to >50** |

Important: For patients enrolled in the USA, an ANC ≥ 0.5 × 10⁹/L (500/μL) and Hb > 8 g/dL (80 g/L) is needed for up-titration. For ANC or Hb values below these thresholds, the current dose is to be maintained or adjusted depending on the platelet count per the above below.
*The DSMC may, upon review of individual patients and patient responses, recommend up- or down-titrations that are not in concordance with the above.
**Upon re-challenge, all of the above rules re-apply.

All patients enrolled in the Phase 1/2A portion of the study, however, required multiple up-titrations of Compound 1 from the original starting dose of 0.25 mg/kg/d to render platelets in the target platelet count range, indicating that the starting dose should be higher. A dose-response curve was subsequently generated that provided a titration algorithm to adjust dose to achieve a target platelet count of between 50,000-75,000 platelets per microliter (k/uL), devised with a view to minimizing the probability of severe thrombocytopenia. Excluding both the highest and lowest doses (total daily doses of 4 mg and 100 mg), the mean total daily dose of Compound 1 needed to achieve a platelet count in the target range was 78.3 mg (S.D. 13.8, range 53-90 mg) or the equivalent of approximately 0.7 to 1.2 mg/kg/d. Accordingly, to enable patients to reach more quickly the optimal dose while still maintaining an adequate safety margin, a new Compound 1 starting dose of 0.5 mg/kg QD was selected for all patients entering the Phase 2B portion of the study. The titration and re-challenge rules were also modified in association with this new target (Table 2).

TABLE 2
Titration and Re-challenge Rules for
the Phase 2b Portion of the Study

| Platelet (Plt Assessment) | | Titration and Re-challenge Rules | | |
|---|---|---|---|---|
| Plt Count | % Plt | | Titration | Re-challenge |
| (×10⁹/L) | Reduction | Titration?* | Rule* | Rule^{¥} |
| ≥90 | <50% from | Up-titrate | Add 0.2 | N/A |
| | previous | | mg/kg/d | |
| | visit^{§} | | | |
| ≥90 | ≥50% from | Up-titrate | Add 0.1 | N/A |
| | previous | | mg/kg/d | |
| | visit^{§} | | | |
| 40-89 | N/A | Maintain | N/A | N/A |
| | | current dose | | |
| 25-39 | N/A | Down-titrate | Decrease current | N/A |
| | | | mg/kg dose by | |
| | | | 25%^{Φ} | |
| <25 | N/A | HOLD | N/A | At 50% of |
| | | DOSE | | previous |
| | | | | dose when |
| | | | | platelets |
| | | | | return |
| | | | | to >50** |

Important: ANC ≥ 0.5 × 10⁹/L (500/μL) and Hb > 8 g/dL (80 g/L) are needed for up-titration. For ANC or Hb values below these thresholds, the current dose should be maintained or adjusted depending on the platelet count per the table below.
*The DSMC may recommend up- or down-titrations that are not in concordance with the above.
**Re-challenge at 50% of the previous mg/kg dose.
^{¥}Upon re-challenge, all of the above rules reapply.
^{§}Note if a platelet count increases since the previous visit the “<” rule should be followed.
^{Φ}Administer 75% of the previous mg/kg dose, which reflects a 25% dose reduction.

Subsequent to amending the dosing algorithm, a re-analysis of dosing, response and safety was conducted based on the experience with the first sixteen patients. The mean dose needed to achieve and safely maintain a patient in the range of the target platelet count (the “therapeutic dose”) was 63.8 mg/d or 0.85 mg/kg/d (assuming an average weight of 75 kg). (Three patients never achieved the target range; two withdrew before week 6 and one did not consent to increasing doses because of fatigue.) Excluding one patient who was maintained on a total daily dose of 4 mg from Day 321 to Day 510, and a second who discontinued the study at Day 35, the range of the total daily therapeutic dosing was 50 mg to 85 mg. In a Phase 3 study, the starting dose is anticipated to be 40 mg with one or two additional dose adjustments made in the subsequent 4-6 weeks.

Eighteen patients enrolled in the Phase 1b/2a portion of the study. Of these, four withdrew early form the study: 1 with progression to accelerated phase disease (Day 39); 2 due to adverse events, fatigue (Day 33), cellulitis (deemed unrelated) (Day 77); and 1 pursuing alternate therapy due to anemia (Day 77). This left 14 patients evaluable for response at week 12, and 9 patients evaluable for response at week 24 so far. An additional 13 have enrolled in the Phase 2b portion, described below. Patient characteristics for the 31 total patents to date are given below in Table 3.

TABLE 3

| | |
|---|---|
| Median Age | 66 (range 48-89) |
| Male/Female | 58%/42% |
| Disease subtype: | |
| PMF | 48% |
| Post-ET MF | 33% |
| Post-PV MF | 19% |
| Risk classification: | |
| High risk | 48% |
| Intermediate-2 risk | 52% |
| Spleen length | Median 23 cm (range: 12-28) |
| Spleen volume | Median 1353 cm³ (range: 192-6819 cm³) |
| Symptom score | Median: 314 (range: 1-82) |
| (MPN-10) | |
| Blood Counts: | |
| WBC | 17.3 × 10⁹/L (range: 1-71) |
| Hemoglobin | 9.5 g/dL (range: 7.2-13.0) |
| Platelets | 197 × 10⁹/L (range: 102-1572) |

All but one patient had received one or more prior treatments including ruxolitinib. 48% had PMF, 33% PET-MF, 19% PPV-MF. The median patient age was 65 years (48-89) with 58% males. 48% were classified as high risk (IPSS), the remainder, intermediate risk-2. Of those that have had a deep genetic analysis (exome sequencing of 264 AML and MPN genes), 71% had more than one mutation, of which 63% were high molecular risk (ASXL1, U2AF1, SRSF2) mutations; 31% had abnormal karyotypes. A substantial fraction of the patients had ≥3 mutations. Patients were treated daily for 12 weeks, in accordance with above starting dose and titration rules, followed by a washout period of up to 28 days. Starting platelet counts ranged from about 141 to about 1309 k/μL. Bone marrow biopsies and imaging studies of the abdomen were conducted prior to treatment and during the washout period after 12 weeks of dosing. Grading of myelofibrosis was performed centrally using the 2016 revised World Health Organization classification of myeloid neoplasia (Arber et al., 2016); image reading was also performed centrally. The Myeloproliferative Neoplasm Symptom Assessment Form (MPN-SAF) was self-administered at Baseline and weekly from Day 0 through the End of Study (EoS) Visit. Total symptom scores were derived from this tool. Patients for whom clinical benefit was demonstrable could resume treatment for additional 12-week cycles.

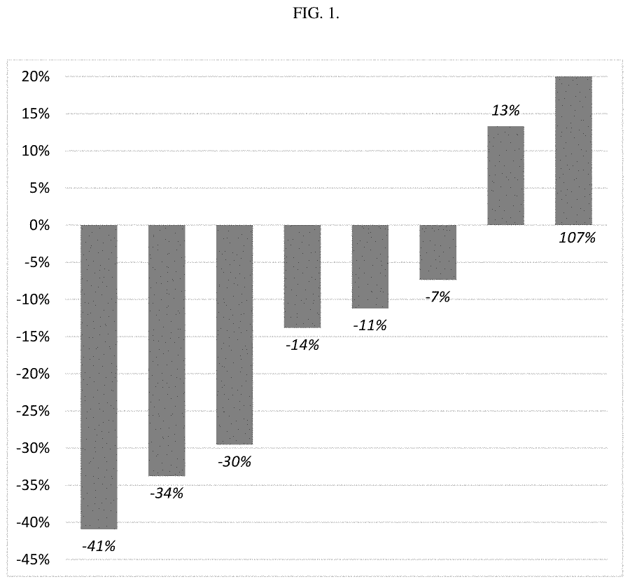

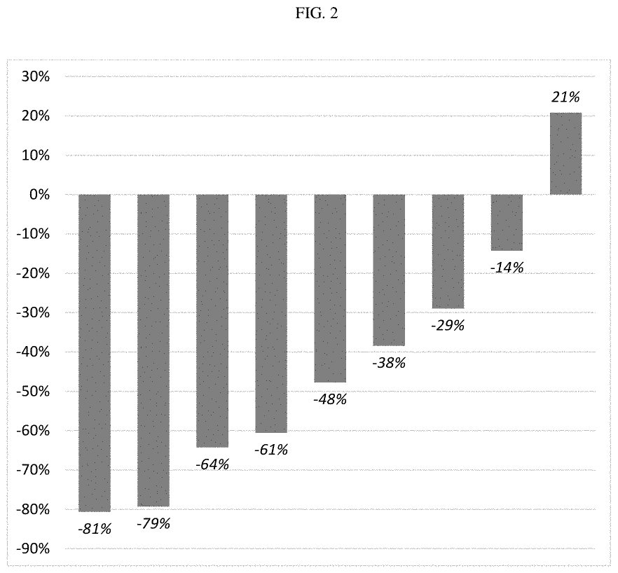

Results. 78% (N=14) of the 18 patients completed 12 weeks (84 days), and 44% (N=9) completed 24 weeks. In the patients deemed evaluable for this preliminary analysis (N=14, those who completed the 85-day cycle and had imaging studies obtained within the first 2 weeks of washout available), Compound 1 had a profound effect on myelofibrosis symptoms. Spleen volumes generally decreased in the patients evaluated so far, as shown in FIG. 1. At week 12, 7 (50%) had a decrease in spleen volume; at week 24, 6 (75%) had a decrease in spleen volume, 1 (12.5%) of these by 35%. MPN-10 scores also generally decreased, as shown in FIG. 2. At week 12, 11 (79%) had reduction in symptom score, 3 of these (21%) by ≥50%; at week 24, 8 (89%) had a decrease in symptom score, 4 (44%) of these by ≥50%.

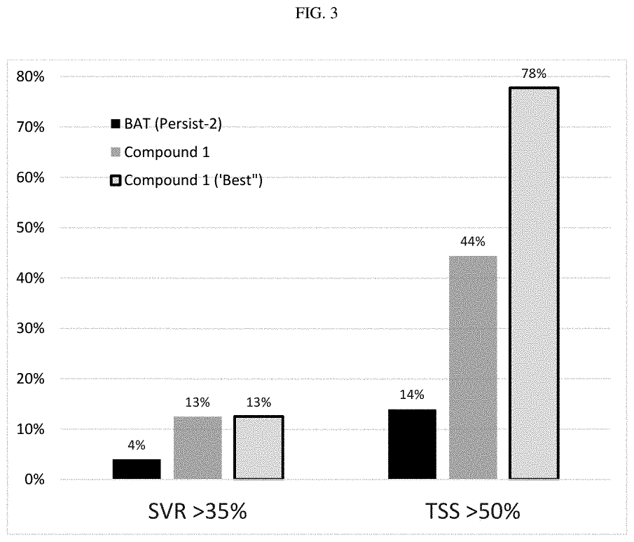

When compared with Best Available Treatment (BAT), e.g., as in the PERSIST-2 clinical trial (see, e.g., clinical trial no. NCT02055781), Compound 1 outperformed the BAT, as shown in FIG. 3: spleen volume response (SVR) and total symptom score (TSS) were both better.

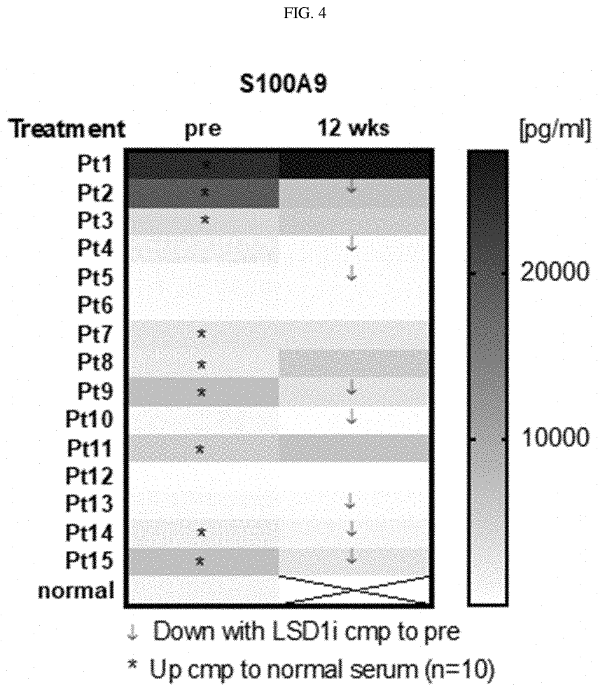

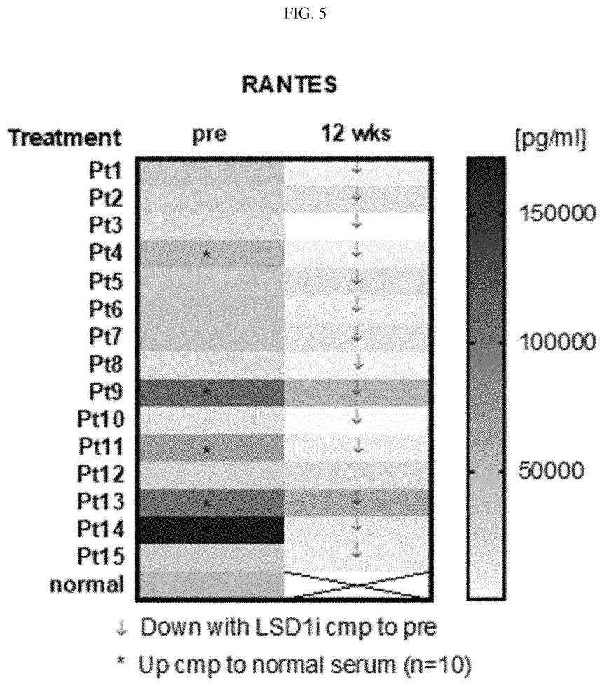

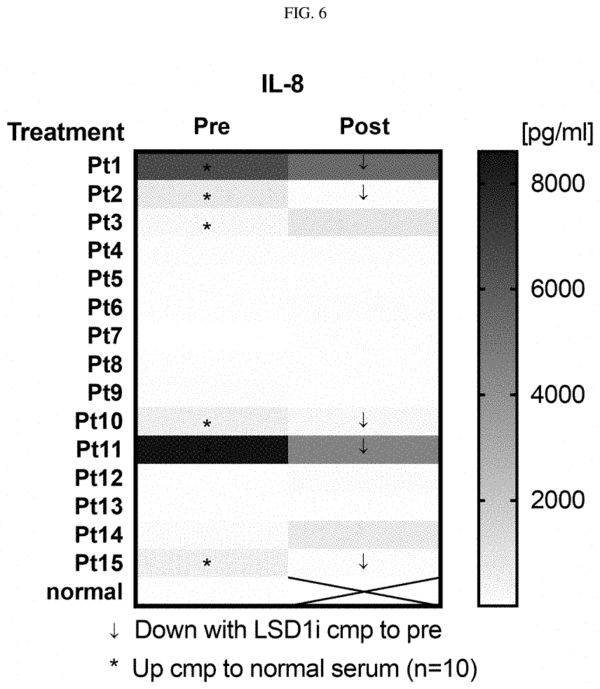

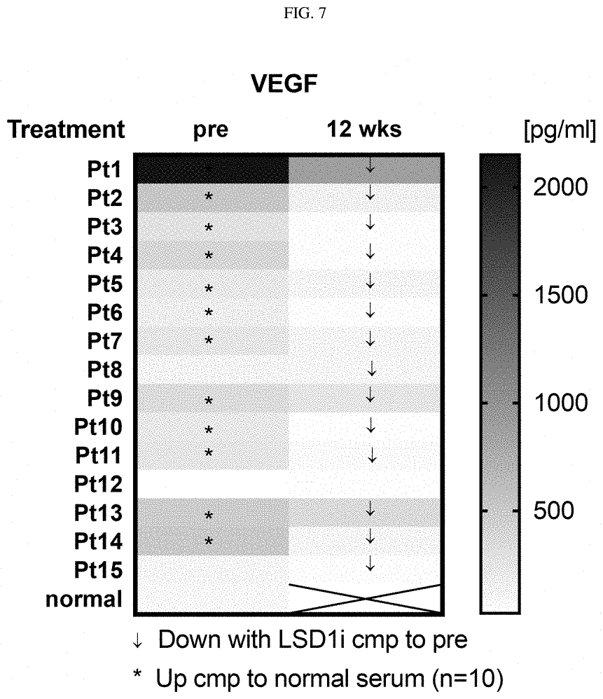

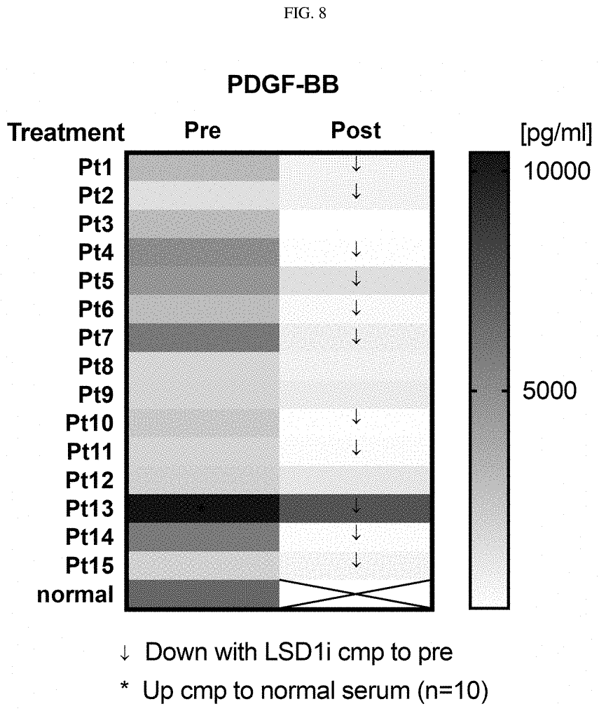

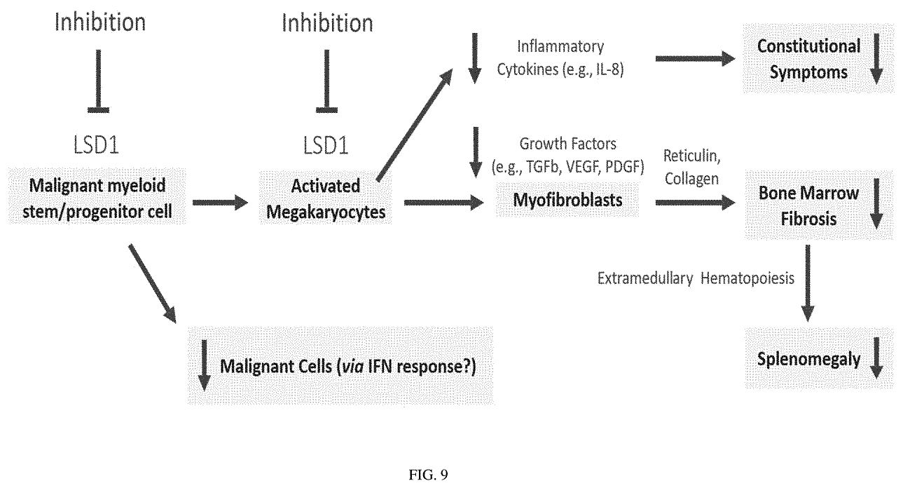

In addition, down-regulation of inflammatory cytokines and reduction in circulating growth factors was observed. As shown in FIGS. 4-6, S100A9 (FIG. 4), RANTES (FIG. 5), and IL-8 (FIG. 6) were generally decreased at week 12 in the course of treatment with Compound 1; meanwhile, levels of CCL3, IL-6, IL-10, IL-33, IL-28A, IFNβ, IFNα, IFNγ were not elevated in any patient. As shown in FIGS. 7 and 8, levels of growth factors VEGF and PDGF-BB were generally decreased at week 12. The relevance of these results in a therapeutic theory of LSD1 inhibition is shown in FIG. 9.

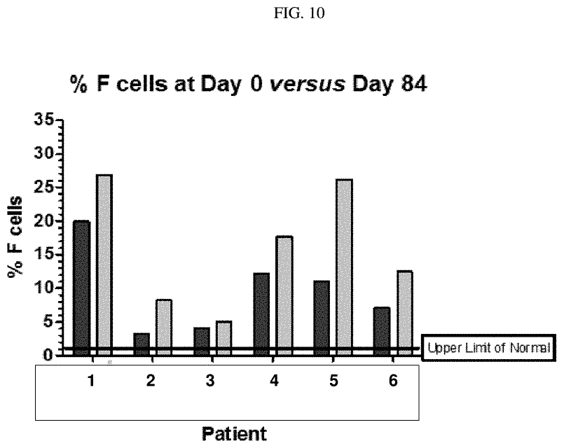

Improvements in hemoglobin (Hb) levels and percent fetal hemoglobin containing erythrocytes (F-cells) were also observed. Of 18 patients enrolled in Phase 1b/2a, 3 presented at day 0 with Hb>10 g/dL and 15 had grade 2 or 3 anemia, with Hb<10 g/dL. Of the 3 with Hb>10 g/dL, 1 improved (defined as an increase of Hb>1 g/dL), 2 worsened (a decrease of Hb>1 g/dL) at day 84 in the course of treatment with Compound 1. Of the 15 patients with Hb<10 g/dL, 9 were transfusion-dependent, and 6 transfusion-independent. Of the 9 transfusion-dependent patients, at day 84, 1 became transfusion-independent and had improved Hb of >1 g/dL, 8 had maintained stable transfusion frequency, and one had increased transfusion frequency. Of the 6 transfusion-independent patients, 1 improved, 3 remained stable, and 2 worsened (1 became transfusion dependent; 1 experienced Hb drop by >1 g/dL. At the same time, Compound 1 reduced the percent of F-cells, as shown in FIG. 10 (in which patients are arbitrarily numbered and do not necessarily correspond to patient numbering in previous figures). Fetal hemoglobin (HbF) is an established serological indicator of cancer, and fetal hemopoiesis, which does not occur in the spleen of healthy adults, has been observed in the spleen in myeloproliferative neoplasms.

Changes in bone marrow fibrosis grade were also observed. Of the 13 patients to date with reported bone marrow biopsies (Day 0 to Day 84 or EoT), 2 (15%) had improvements of ≥1 grade, 8 (62%) had a stable fibrosis score, and 3 (23%) progressed by 1 Grade.

With respect to symptom scores, improvements were generally dose-dependent and rapid, e.g., scores of fatigue improved in 8 of the first 16 patients within 14 days. These changes were observed at what represented the lowest two doses for all but one of these 16 patients. Similar to what has been reported in studies of JAK inhibitors in patients with MF, there was no correlation between improvements in symptomatology and changes in spleen volume. The reductions in spleen volumes were compromised in several ways. Patients were deliberately treated with an initial dose that was expected to be sub-optimal and most did not achieve a platelet count in the target range until halfway into the 85 day cycle. Further, all patients had follow-up imaging studies during the washout period—it became readily apparent by physical exam that spleen volumes increased. In the Phase 2b portion of the study, the washout period has been eliminated and the dosing regimen has been improved to more rapidly achieve the target platelet count and to maintain the patient in that range safely for longer.

Treatment with Compound 1 reduced platelet counts in all patients. The change in platelet production was tightly associated with exposures to Compound 1—platelet counts could be titrated with reasonable precision. The kinetics of these changes were congruent with the known life span of a human platelet—7 days. With the cessation of treatment, platelet counts rebounded robustly indicating the reversibility of the anti-thrombopoietic effect of Compound 1 once drug has cleared. As with rat and dog, granulocyte production appeared less sensitive to LSD1 inhibition; peripheral granulocyte counts were lower on treatment, lymphocytes counts were unchanged and monocyte counts were generally modestly elevated. These observations are consistent with what was observed in both the non-clinical studies and in the other clinical studies.

Safety. Throughout the course of the study, there were no deaths or dose-limiting toxicities occurred. Four SAEs attributed to Compound 1 (all Grade 3), including painful splenomegaly, headache, nausea and vomiting, and heart failure. There were 139 AEs of all grades attributed to Compound 1. The most common AEs across the 31 subjects from both of the above studies were thrombocytopenia (11 subjects, 35%), anemia (3 subjects, 10%) and nausea (1, 3%). The most common grade 3/4 AEs attributed to Compound 1 were Anemia (6 subjects, 19%) and neutropenia (3 subjects, 10%).

The foregoing demonstrates that in a heterogeneous population of patients with MF with limited therapeutic options, Compound 1 was well tolerated, appeared safe and was effective in reducing spleen volumes and substantially improving symptom scores in a majority of patients.

### Example 2: Phase 2B Clinical Trials in Myelofibrosis

A multi-center, open label, dose-range finding study to assess the safety, optimally effective dosing rules, steady-state pharmacokinetics and pharmacodynamics of Compound 1 orally once daily in patients with myelofibrosis was conducted in a Phase 1/2a study.
Primary objectives were to evaluate, in MF patients the effect of Compound 1 on: Safety and tolerability
Pharmacokinetics (Phase 1/2a only)
Reduction in spleen volume

Exploratory Objectives (some or all may be analyzed) included evaluating, in MF patients treated with Compound 1:
The adequacy of the treatment regimen in producing a pharmacodynamic effect
Hematologic response (Hematologic parameters, all of which may be assessed during treatment or after drug has been discontinued for a specified interval, may comprise: complete blood count (CBC) including platelets, red and white blood cell (RBC and WBC) and circulating blast cell counts; cellular composition of the bone marrow (% blasts); and, the induction of fetal hemoglobin)
Improvement in constitutional symptoms assessed using the Myeloproliferative Neoplasm Symptom Assessment Form (MPN-SAF)
Reduction in bone marrow fibrosis score
Relationship between dose and plasma trough concentrations over time (Phase 1/2a only)
The impact of therapy on disease burden as measured by malignant-cell specific nucleic markers (DNA or RNA; nucleic markers include RNA and/or DNA mutations detected by sequencing or other nucleic assay methods)
The effect of treatment on cytokine profiles (cytokine quantification)
The relationship between genetic aberrations in malignant cells and pharmacodynamic response
And to correlate conventional clinical responses with exploratory assessments of response

Compound 1 was supplied as capsules in multiple strengths. These strengths, based on Compound 1 free base, i.e., the active substance, may include: 1 mg, 5 mg, 10 mg, 25 mg and 50 mg. Capsule strengths provided may change throughout the duration of the study.

The therapeutic goal for the treatment of MF was to inhibit the activity of LSD1 in hematopoietic cells for only a portion of the 24-hour dosing cycle, sufficient to reduce the production of cytokines and growth factors that drive bone marrow fibrogenesis. Considerations for a safe and therapeutic starting dose included chronic toxicology studies, in conjunction with the clinical experience of the patients who have received Compound 1 to date in prior studies. In association with this therapeutic goal, and PK modeling, a starting dose (Ds) of 0.25 mg/kg/d was selected for the Phase 1/2A portion of this study. All patients, however, required multiple up-titrations of Compound 1 from this starting dose to render platelets in the target platelet count range, suggesting the Ds should be higher. A dose-response curve was subsequently generated that provided a titration algorithm to adjust dose to achieve a target platelet count of between 50,000-75,000 platelets per microliter (k/uL), devised with a view to minimizing the probability of severe thrombocytopenia. Excluding both the highest and lowest doses, the mean total daily dose of Compound 1 needed to achieve a platelet count in the target range was 78.3 mg (S.D. 13.8, range 53-90 mg) or the equivalent of approximately 0.7 to 1.2 mg/kg/d. Accordingly, to enable patients to more quickly reach the optimum dose while still maintaining an adequate safety margin, a new Compound 1 starting dose of 0.5 mg/kg QD was selected for all patients entering the Phase 2b portion of the study.

This study design used the alternative model-based approach appropriate for a targeted, non-cytotoxic drug such as Compound 1 in which there is no observed monotonic relationship between exposure and toxicity (Le Tourneau, et al., 2009). Specifically, this study employed the dose-toxicity model developed in rat and dog relating the plasma concentration of drug 24 hours after last dose (Cₘᵢₙ) at steady state needed to inhibit platelet production.

As there is no evidence in non-clinical studies of acute toxicity with Compound 1, even at extremely high doses (human equivalent dose (HED) ˜20-40 mg/kg), it was believed that two sentinel patients would be sufficient to establish the acute safety of the starting dose. Thus, two sentinel patients were dosed sequentially at the original Ds of 0.25 mg/kg/d for 7 days and monitored twice-weekly before any additional patients were treated. Since this study was not investigating the effect of a cytotoxic agent, enrolling patients on a rolling basis post-establishing safety via dosing of the sentinel patients was deemed appropriate. Patients were enrolled and treated on a rolling basis.

To ensure patient safety, a Data Safety Monitoring Committee (DSMC) performed monthly reviews of safety parameters and pharmacodynamic markers to draw conclusions around the safety and pharmacodynamic effect of Compound 1. The DSMC also reviewed patient dose titrations and recommended dose adjustments, and assessed the necessity of the Day 3 visit. The DSMC convened within 4 days post-completion of 7 days of treatment for each of the sentinel patients and determined it was safe for:

1. Each sentinel patient to continue dosing (note: dosing was not interrupted pending this review), and
2. Additional patients to begin treatment with Compound 1.

### Study Conduct

This study initiated as a Phase 1/2a study assessing the safety of the starting dose, an 85 day duration of treatment, and the pharmacokinetic and pharmacodynamic effects of Compound 1, with transition to a Phase 2b study incorporating changes supported by the earlier pharmacokinetic and pharmacodynamic studies and safety assessments. This study consisted of two treatment periods: the Initial Treatment Period (ITP), followed by the Additional Treatment Period (ATP). Patients commenced enrolment in the Phase 2b portion of the study, in which the ITP has been extended such that patients were treated daily for 169 days. The ATP, also extended, offered treatment to qualifying patients for an additional 169 days.

Initial Treatment Period. During the ITP, patients initially returned for study assessments twice weekly for the first week (ITP Days 0, 3 and 7); post-dosing of 3 patients at the new Ds, the DSMC convened to assess the necessity of the Day 3 visit. Patients returned weekly for the next 7 weeks (ITP Days 14, 21, 28, 35, 42, 49 and 56), at least bi-weekly for 8 weeks (ITP Days 70, 84, 98 and 112) and then monthly for 8 weeks (ITP Days 140 and 168). It was anticipated that by Week 8 (Day 56) patients will have achieved a stable dose, with weekly titrations no longer necessary. For the exceptional patient whose dose had not stabilized, weekly visits continued at the PI's discretion (note: bi-weekly visits may also continue post Day 112). On Days 84 and 168, patients underwent abdominal magnetic resonance imaging (MRI), or computerized tomography (CT) if the patient was not a candidate for MRI. On Day 168, bone marrow sampling was also required. Prior to or at the Day 168 visit, but ideally at the Day 140 visit for logistical purposes, a ‘qualification’ assessment was made to determine whether the patient is deriving clinical benefit (defined as not meeting progressive disease criteria and safely tolerating Compound 1; this definition applies throughout document and will not be repeated with each reference to clinical benefit). Such patients qualified for entry into the ATP, a transition which was anticipated to occur without interruption in dosing. Patients not deriving clinical benefit, or who achieve complete response (CR), partial response (PR) or clinical improvement (CI) and subsequently relapse the equivalent of treatment failures, discontinued Compound 1 and undergo End of Treatment (EoT), pre-End of Study (pre-EoS) and End of Study (EoS) visits.

Additional Treatment Period. In the ATP, treatment was expected to continue for an additional 169 days in those patients deriving clinical benefit, as determined by the Principal Investigator. Qualifying patients returned for study assessments monthly (ATP Days 0, 28, 56, 84, 112, 140 and 168). It was anticipated that patients continuing in the ATP will have already achieved a stable dose, with frequent titrations no longer necessary. For the exceptional patient whose dose had not stabilized, bi-weekly visits continued at the PI's discretion. On Day 168, patients underwent the same procedures and assessments as in the ITP, including MRI or CT (if the patient was not a candidate for MRI), and bone marrow sampling. Prior to or at the Day 168 visit, but ideally at the Day 140 visit for logistical purposes, a ‘qualification’ assessment was made to determine whether the patient is continuing to derive clinical benefit. Such patients thereby qualified for re-entry into the ATP, which is iterative; patients continued to receive Compound 1 for as long as they continued to qualify.

Certain patients enrolled in prior clinical trials with Compound 1 would complete their current Treatment Phase, in accordance with that protocol, prior to initiating the extended ATP disclosed herein. Such patients did not undergo any washout period between Treatment Periods, but the assessments prescribed during washout were still performed with MRI (or CT) and bone marrow aspirate and biopsy required at the Day 84 visit. For these patients, the ‘qualification’ assessment occurred at the study visit immediately preceding the Day 84 visit.

The assessments that were prescribed during the washout were still done despite the elimination of the washout. All patients underwent follow-up period visits, including an EoT visit within approximately 2 days of last dose, a pre-EOS visit approximately 14 days post last dose, and an EoS visit approximately 28 days post last dose. Patients that did not enter the ATP, or discontinue early, entered the follow-up period beginning with an EoT visit within approximately 2 days of the decision to end treatment.

Patients were followed closely throughout the study for both Adverse Events (AEs) and signs of toxicity by frequent monitoring of clinical signs and symptoms and by peripheral blood and urine analyses. Pharmacodynamic effects were closely monitored by frequent hematology assessments of peripheral blood, and requisite bone marrow aspirates and biopsies. Throughout dosing, transfusions were administered if needed in accordance with standard institutional guidelines.

### Dosing

Through the use of dose titration, all patients were dosed to the estimated dose of Compound 1 needed in humans that provides sufficient exposure to inhibit normal hematopoiesis safely for a portion of the 24-hour dosing cycle (designated the Dpi).

Initial Treatment Period (ITP). Treatment began on Day 0 at the Ds of 0.5 mg/kg QD for all patients entering the Phase 2b portion of the study. Dose-adjustments could be made at each clinic visit (with the exception of Day 3), with dose-titration, either upward or downward, contingent on the comparison of hematology values from the prior visit, as dictated by the rules below. The Dpi was anticipated to be ≤1.2 mg/kg QD; however, this was not the upper limit for titration purposes as the dose needed to achieve a therapeutic effect will vary among patients and may change over time. The platelet titration target expected to be associated with a clinically significant therapeutic effect was a platelet count of ≥50,000 to ≤75,000/μL (50-75×10⁹/L). Titration and re-challenge rules based on evaluation of platelet, absolute neutrophil (ANC) and hemoglobin (Hgb) counts are noted below.

Titration rules. Important: ANC ≥0.5×10⁹/L (500/μL) and Hgb >8 g/dL (80 g/L) were needed for up-titration. For ANC or Hgb values below these thresholds, the current dose was maintained or adjusted depending on the platelet count per Table 4 below.

TABLE 4

| Platelet (Plt Assessment) | | Titration and Re-challenge Rules | | |
|---|---|---|---|---|
| Plt Count | % Plt | | Titration | Re-challenge |
| (×10⁹/L) | Reduction | Titration?* | Rule* | Rule¥ |
| ≥90 | <50% from | Up-titrate | Add 0.2 | N/A |
| | previous | | mg/kg/d | |
| | visit§ | | | |
| ≥90 | ≥50% from | Up-titrate | Add 0.1 | N/A |
| | previous | | mg/kg/d | |
| | visit§ | | | |
| 40-89 | N/A | Maintain | N/A | N/A |
| | | current dose | | |
| 25-39 | N/A | Down-titrate | Decrease | N/A |
| | | | current mg/kg | |
| | | | dose by 25%Φ | |
| <25 | N/A | HOLD | N/A | At 50% of |
| | | DOSE | | previous |
| | | | | dose when |
| | | | | platelets |
| | | | | return |
| | | | | to >50** |

*The DSMC may recommend up- or down-titrations that are not in concordance with the above.
**Re-challenge at 50% of the previous mg/kg dose.
¥Upon re-challenge, all of the above rules reapply.
§Note if a platelet count increases since the previous visit the “<” rule should be followed.
ϕAdminister 75% of the previous mg/kg dose, which reflects a 25% dose reduction.

Dose reductions could be made at any time in consultation with the Medical Monitor, should an AE requiring a dose reduction occur.

Additional Treatment Period (ATP): Qualifying patients would ‘re-start’ Compound 1 on ATP Day 0, with dose titration continuing as per the Titration Rules table above; there was no interruption in dosing (i.e., Day 168=Day 0 of new ATP). Additional dose-titration could occur in consultation with the Medical Monitor.

Study Duration. Screening procedures could commence up to 28 days prior to the start of treatment. Patients could initially receive up to 169 days of dosing while on study. Patients were followed for 28 days post last dose. Therefore, the anticipated duration of participation in the study was expected to be at least 32 weeks from first patient-first visit (FPFV) to last patient-last visit (LPLV). Additional treatment could be given, contingent on an assessment of patient benefit.

Study Assessments. The assessments outlined below are presented in detail by study visit.

The Myeloproliferative Neoplasm Symptom Assessment Form Total Symptom Score (MPN-SAF TSS) will be completed at Baseline and on each visit day (with the exception of Day 3) from Day 0 through the End of Study (EoS) Visit.

Adverse events (AEs) will be assessed at every visit post first Compound 1 dose through the EoS visit.

Physical Examinations (PE), including vital signs: a Full Physical Exam will be performed at Screening. Limited Physical Exams (LPE) will be performed at all other clinic visits (with the exception of Day 3) throughout the study. LPEs include weight, a review of body systems to assess change from previous PE, and spleen measurement. The edge of the spleen shall be determined by palpation, measured in centimeters, using a soft ruler/tape, from the costal margin to the point of greatest splenic protrusion. The spleen should be measured in the same manner at all visits.

Urine or serum pregnancy testing will be performed for women of child-bearing potential (WOCBP) at Screening, Baseline (if separate from Screening visit), pre-dose Day 0, monthly (i.e., Days 28, 56, 84, 112, 140 and 168) throughout the study, upon suspicion of relapse, at the EoT, pre-EoS, and EoS/ET visits and if pregnancy is suspected while the patient remains on-study.

Bone marrow aspirate and biopsy will be performed:
At Baseline (no more than 21 days prior to the first Compound 1 dose).
At Day 168 (±7 days).
Approximately every 6 months thereafter, at Day 168 (±7 days) of the ATP, for as long as the patient continues to qualify.
At EoT and ET (unless performed within the prior 5 weeks), and upon suspicion of relapse (unless performed in the last 21 days or is scheduled in the next 7 days).
   Aspirate from the first pull whenever possible, but no later than the second pull, is required. The total number of bone marrow evaluations required during the ITP is 2 in ˜32 weeks. Additional marrow evaluation is required only if the patient qualifies for the ATP, demonstrates response followed by suspected relapse, or evidence of progressive disease.

MRI or CT (if the patient is not a candidate for MRI) of the abdomen will be performed:
Pre-dose Day 0 (±2 days)
At the Day 84 and Day 168 visits (±7 days)
Approximately every 6 months thereafter, at Day 168 (±7 days) of the ATP, for as long as the patient continues to qualify
At EoT, ET, and upon suspicion of relapse (unless performed within the prior 5 weeks)

Clinical laboratory measures: The following laboratory measures will be performed at Screening, Baseline (if separate from Screening visit), pre-dose Day 0, upon suspicion of relapse, and at the EoT, pre-EoS, and EoS/ET visits, and in accordance with the below:

Biochemistry—monthly (i.e., Days 28, 56, 84, 112, 140 and 168) throughout the study

Hematology with manual differential—every clinic visit throughout the study

Coagulation—monthly (i.e., Days 28, 56, 84, 112, 140 and 168) throughout the study

Urinalysis—Day 84 and Day 168 throughout the study

Cytokines: Sample collection time-points are below.
Pre-dose Day 0, Days 14, 28, 84 and 168, and each Day 168 visit of the ATP, for as long as the patient continues to qualify
At EoT, and at ET (ET required only if the patient discontinues during the ITP)

Red Cell Hemoglobin F (HbF) and % F cells (Selected sites only/ITP only):

Pre-dose Day 0, Day 84 and Day 168

At EoT and ET (both required only if the patient discontinues during the ITP)

Genomic analysis: Germline samples should be collected at Baseline; however, may be collected up to and including pre-dose Day 1. Repeat sampling may be necessary, pending sample yield.

Blood samples will be collected for genomic analysis at the following time-points:
At Baseline (no more than 21 days prior to the first Compound 1 dose)
At the Day 84 and Day 168 visits
Approximately every 6 months thereafter, at each Day 168 visit of the ATP, for as long as the patient continues to qualify
At EoT, EoS/ET and upon suspicion of relapse Any bone marrow aspirate samples will undergo genomic analysis as per the bone marrow sampling schedule.

Pharmacodynamic (PD) Assessments: PD parameters will be assessed using blood and bone marrow samples collected both during treatment and after treatment has been discontinued for a specified interval. The following may be performed: a complete blood count (CBC) with white blood cell differential; measurement of circulating cytokines; and, measurement of RNA and/or DNA mutations and their frequencies identified by sequencing; and, the induction of fetal hemoglobin. A bone marrow evaluation, including morphology and fibrosis score will be performed in association with every bone marrow sampling time-point.

Eligibility Criteria. Patients must meet all applicable Inclusion and none of the Exclusion Criteria.

Inclusion Criteria:
1. Informed consent.
2. Age: 18+ years old at Screening.
3. Diagnosis of either PMF per World Health Organization (WHO) diagnostic criteria for myeloproliferative neoplasms, PPV-MF per the IWG-MRT, or PET-MF per the IWG-MRT and meet the following additional subtype specific criteria:
   a. Classified as high risk (3 prognostic factors) OR intermediate risk-2 (2 prognostic factors). The prognostic factors, defined by the International Working Group (Cervantes, et al., 2009):
      i. Age >65 years;
      ii. Presence of constitutional symptoms (weight loss, fever, night sweats);
      iii. Marked anemia (Hgb <10 g/dL) (hemoglobin value <10 g/dL must be demonstrated during Screening for patients who are not transfusion dependent. Patients receiving regular transfusions of packed red blood cells will be considered to have hemoglobin <10 g/dL for the purpose of evaluation of risk factors.);
      iv. History of leukocytosis [WBC >25×10⁹/L (25,000/μL)];
      v. Circulating blasts >1%.
4. Be refractory or resistant to, inadequately controlled by or intolerant of available approved therapy, or in the Investigator's judgment, are not candidates for available approved therapy (note: approved therapy includes ruxolitinib).
5. Eastern Cooperative Oncology Group (ECOG) performance status score ≤2.
6. Peripheral blast count ≤10% prior to dosing on Day 0.
7. Absolute neutrophil count ≥0.5×10⁹/L (500/μL) prior to dosing on Day 0.
8. Platelet count ≥100×10⁹/L (100,000/μL) prior to dosing on Day 0.
9. Life expectancy >36 weeks.
10. Have discontinued all previous therapies for MPNs including ruxolitinib, any chemotherapeutic agents, immunosuppressive therapy (e.g., corticosteroids >10 mg/day with the noted exception: use of corticosteroids for management of gout is allowed; maintenance supplemental corticosteroid therapy such as prednisone ≤10 mg/day or corticosteroid equivalent is allowed), immune modulators (e.g., thalidomide), radiotherapy for at least 2 weeks prior, and interferon for 4 weeks prior to study Day 0. Low dose acetylsalicyclic acid is permitted. Palliative radiation treatment to non-index or bone lesions performed <2 weeks before treatment may be considered with Medical Monitor approval.
11. Amenable to bone marrow evaluation, peripheral blood and urine sampling during the study.
12. Able to swallow capsules.
13. Women of childbearing potential (WOCBP) and fertile men must agree to use an approved method of contraception from Screening until 28 days after last Compound 1 dose. Methods of contraception include: estrogen and progestogen combined hormonal contraception which inhibits ovulation; progestogen-only hormonal contraception associated with inhibition of ovulation; intrauterine device (IUD); bilateral tubal occlusion; vasectomized partner in a monogamous sexual relationship (vasectomy or tubal ligation at least six months prior to dosing); and, complete sexual abstinence (defined as refraining from heterosexual intercourse). Patients practicing abstinence must agree to use an approved method of contraception should they become sexually active during the study. The risk of embryofetal toxicity is fully mitigated by 28 days which is >10 half-lives of the drug at the doses used in this study.

Exclusion Criteria:
1. Has undergone major surgery ≤4 weeks prior to starting study drug or has not recovered from side effects of such surgery.
2. Has undergone any surgical procedure within 2 weeks, excluding minor procedures (e.g., skin biopsy or central venous catheter placement/removal) prior to starting study drug.
3. History of splenectomy.
4. History of or scheduled hematopoietic stem-cell transplant within 24 weeks of screening.
5. Unresolved treatment related toxicities from prior therapies (unless resolved to ≤Grade 1).
6. Current use of a prohibited medication (e.g., romiplostim) or expected to require any of these medications during treatment with the investigational drug.
7. Known immediate or delayed hypersensitivity reaction or idiosyncrasy to drugs chemically related to Compound 1 or LSD1 inhibitors (i.e., monoamine oxidase inhibitors; MAOIs) that contraindicates their participation.
8. Current use of monoamine oxidase A and B inhibitors (MAOIs).
9. Uncontrolled active infection.
10. A concurrent second active and non-stable malignancy (patients with a concurrent second active but stable malignancy, such as non-melanoma skin cancers, are eligible).
11. Evidence at the time of Screening of risk of bleeding, including any of the following:
   a. Activated partial thromboplastin time (aPTT) ≥1.3× the local upper limit of normal
   b. International normalized ratio (INR) ≥1.3× the local upper limit of normal
   c. History of severe thrombocytopenia or platelet dysfunction unrelated to a myeloproliferative disorder or its treatment
   d. Known bleeding disorder (e.g., dysfibrinogenemia, factor IX deficiency, hemophilia, Von Willebrand's disease, Disseminated Intravascular Coagulation [DIC], fibrinogen deficiency, or other clotting factor deficiency)
12. Evidence at the time of Screening of significant renal or hepatic insufficiency (unless due to hemolysis, or leukemic infiltration) as defined by any of the following local lab parameters:
   a. Calculated glomerular filtration rate (GFR; using the Cockcroft-Gault equation) <40 mL/min or serum creatinine >1.5× the local upper limit of normal
   b. Aspartate transaminase (AST) or alanine aminotransferase (ALT) ≥2× the local upper limit of normal
13. Known human immunodeficiency virus (HIV) infection or known active Hepatitis B or Hepatitis C virus infection (testing will not be conducted as part of Screening procedures).
14. History of any illness/impairment of gastrointestinal (GI) function that might interfere with drug absorption (e.g., chronic diarrhea), confound the study results or pose an additional risk to the patient by participation in the study; patients with gastric bypass surgery.
15. Use of an investigational agent within less than 14 days, or the equivalent of at least 7 half-lives of that agent, whichever is the longer, prior to study Day 0.
16. Pregnant or lactating females; females intending to become pregnant at any time during the study.

Safety Guidelines. In general, supportive care (transfusions, administration of anti-fungals, etc.) should be maintained in accordance with institutional policy. Additionally, it is advised that patients with a platelet count ≤10×10⁹/L (10,000/μL) be transfused. Hydroxyurea may be used during the study in case of proliferation: a) at the primary investigator's discretion, initiate hydroxyurea treatment for white cell count ≥30×10⁹/L (30,000/μL) and majority of cells appear to be immature cells (myelocytes/promyelocytes); and b) discontinue hydroxyurea treatment when white cell count is ≤10×10⁹/L (10,000/pL).

Patients taking medications that have the potential to induce or inhibit CYP3A4 or CYP2D6 should be monitored closely for potential effects of co-administration; particular attention should be given to anti-infectives in the azole class.

Prohibited Medications/Treatments.
1. All cytotoxic agents, with the exception of hydroxyurea
2. Thromobopoietic agents: romiplostim, eltrombopag
3. Prednisone or prednisolone >10 mg/day (noted exception: use of corticosteroids for management of gout is allowed) and dexamethasone >4 mg/day. Maintenance supplemental corticosteroid therapy such as prednisone ≤10 mg/day or corticosteroid equivalent is allowed.
4. Monoamine oxidase A and B inhibitors
5. Anticoagulant and nonsteroidal anti-inflammatory drug (NSAID; including aspirin) use are prohibited in patients when their platelet count is <50×10⁹/L (50,000 μL).
   LSD1 inhibition may induce cytopenias which, in turn, may cause an increase in granulocyte and granulocyte-macrophage colony stimulating factor (G-CSF and GM-CSF) and erythropoietin (EPO). Though not expressly prohibited, G-CSF, GM-CSF and EPO given exogenously are not likely to be of clinical benefit in the setting of granulocytopenia or anemia, respectively, secondary to inhibition of LSD1.

Management of Study Toxicities. Adverse event intensity will be evaluated using the National Cancer Institute (NCI) Common Terminology Criteria for Adverse Events (CTCAE) version 4.03, published 14 Jun. 2010.

Hematologic Toxicity: Hematologic values outside of the normal reference range are inherent features of MPNs, and are expected effects of many therapeutic attempts to manage these diseases. The effects of Compound 1 on normal myeloid hematopoiesis observed in non-clinical and clinical studies are expected in humans; these are pharmacodynamic effects of LSD1 inhibition by Compound 1, thus not regarded as adverse. These events, with the exceptions below, will not be considered DLTs.

Dose limiting toxicity (DLT): Any one of the following AEs that occurs through Day 7 of the Initial Treatment Period and is considered by the Investigator to be possibly, probably or definitely related to Compound 1:
Thrombocytopenia leading to clinically significant sequelae (i.e., a clinically significant bleeding event* or the need for prophylactic transfusions);
A clinically significant bleeding event in a patient with a platelet count >50,000×10⁹/L (50,000/μL), wherein a clinically significant bleeding event is defined as an event that is life-threatening, cannot be controlled and/or results in hemodynamic instability;
   Any Grade 4 or 5 non-hematologic adverse event;
   Any Grade 3 non-hematologic adverse event with failure to recover to Grade 2 within 7 days of drug cessation, with the following exceptions:
≥Grade 3 nausea, vomiting or diarrhea that responds to standard medical care
≥Grade 3 aesthenia lasting less than 14 days
Any Grade 3 electrolyte abnormality unrelated to the underlying malignancy and persisting greater than 24 hours.
   Patients who experience a DLT may have their dose adjusted downward if it is deemed safe for the patient to continue on Compound 1.

Stopping Rules. Treatment will be discontinued if: post DLT, it is deemed unsafe for the patient to continue on Compound 1; post dose reduction due to DLT, the patient fails to demonstrate significant improvement within 21 days; or post temporary interruption of Compound 1 due to platelet counts below 25×10⁹/L (25,000/μL), the patient's platelet counts do not return to >50×10⁹/L (50,000/μL) within 21 days.

Results. 13 patients enrolled in the study; 85% remain on study so far.

All patients at Week 12 are characterized by the following:

Total symptoms (n=32)
78% (25) had a decrease in symptom score
25% (8) had a reduction of ≥50%

Phase 2b patients at Week 12 are characterized by the following:

Spleen Volume (n=14)
86% (12) had a decrease in spleen volume
14% (2) had a reduction of ≥35%
29% (4) had a reduction of ≥20%
Median change to Wk 12=−15%

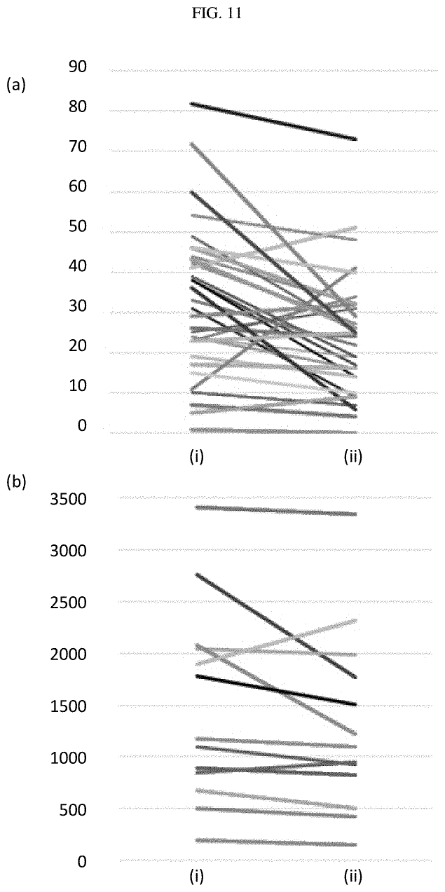

Absolute change in MPN SAF TSS and spleen volume over the course of 12 weeks is shown in FIGS. 11(a) and (b), respectively.

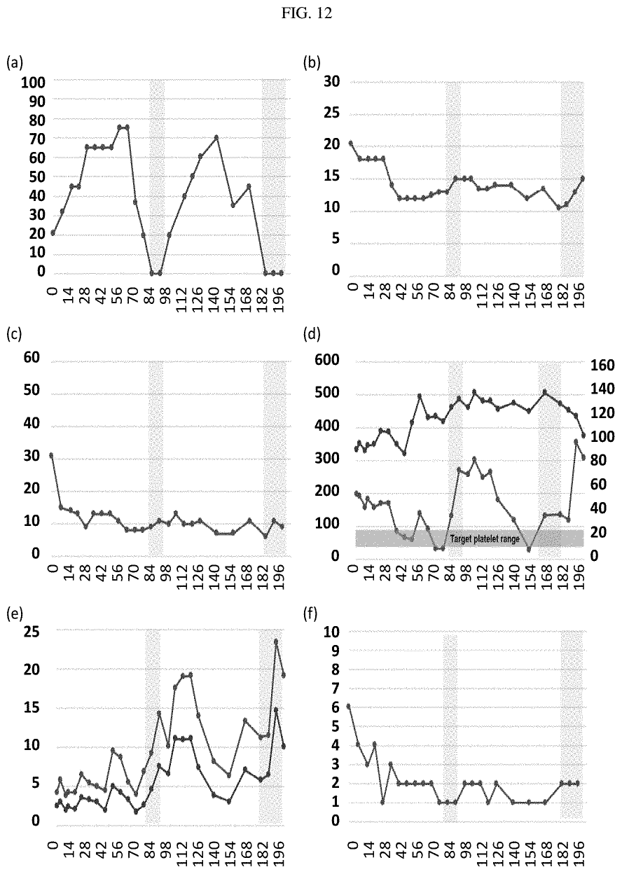

FIG. 12 shows the progress of treatment for patient 008-103, over a course of 196 days. Dosage titration of LSD1 inhibitor, in mg, is shown in panel (a). The effect of this dosage regimen is shown in the following panels: (b) spleen size, cm; (c) symptoms score; (d) platelets (left scale, k/uL) and hemoglobin (right scale); (e) WBC and neutrophils; and (f) fatigue score (10=worst).

### Example 3: Sequencing Protocol

The following characterize the sequencing protocol:
Samples: Germline (buccal or hair) and “Tumor” (bone marrow, peripheral blood, granulocytes)
Target enrichment: 11,736 hybridization probes in IDT AML panel targeting 261 genes (˜6300 exons) recurrently mutated in myeloid neoplasms
Illumina sequencing: 2×150 bp paired-end sequencing; ˜10 million pairs sequenced per sample
Aiming for sequencing depth >500; Actual: >1000 for >90% of samples
Analysis: Burrows-Wheeler alignment (BWA)=>VARSCAN2 genotyper=>IGV for CALR, etc.
Cutoffs for somatic calls: Sequencing Depth: >20 Mutant (or Variant) Allele Frequency (VAF): >15%
Annotation: All calls submitted to CADD (Combined Annotation Dependent Depletion) at University of Washington
   CADD score cutoff >20 identifies the top 1% of the most deleterious mutations

The following were observed:

7/22 (32%) show a decrease in some or all somatic mutations

12/22 (55%) have stable VAFs

3/22 (14%) patient have an increased VAFs

No new mutations identified in patients followed up to 550+ days

No progression to AML

The following table presents both MPN somatic mutations and other somatic mutations, as well as VAF at follow up

| Patient ID | MPN somatics | Other somatics | VAF @ follow up |
|---|---|---|---|
| 003-101 | JAK2_V617F | U2AF1_Q157R | Stable |
| 006-101 | JAK2_V617F | ZBTB33_Y56S | Partial Improvement |
| 006-102 | JAK2_V617F | | Stable |
| 007-104 | CALR_52b_del | ASXL1_-642X | Stable |
| 008-101 | MPL_W515K | ASXL1_Q780* | Partial Improvement |
| 008-102 | JAK2_V617F | TET2_NRN1890- | Stable |
| 008-103 | CALR_K385NCX | ASXL1_R693* | Partial Improvement |
| 008-105 | JAK2_V617F | ASXL1_-884X PRPF8_R1832C | Stable |
| 010-102 | CALR_52b_del | CBL_C396S ASXL1_-642X | Partial Increase |
| | | EZH2_-262X | |
| 010-103 | JAK2_V617F | CBL_R420Q EZH2_F145L | Stable |
| | | CNTN5_P220L ASXL1_QLL695HX | |
| 010-104 | JAK2_V617F | SF3B1_K700E DNMT3A_V687G | Improvement |
| | | TET2_S1284F | |
| 010-105 | JAK2_V617F | DNMT3A_V687G | Stable |
| 011-101 | MPL_W515K | | Increase |
| 011-102 | JAK2_V617F | ASXL1_Q768* PRPF8_D1598V | Stable |
| | | FREM2_S204R | |
| 011-104 | JAK2_V617F | MAP1B_D1587N ASXL1_-642X | Stable |
| 011-105 | JAK2_V617F | ASXL1_HHCHREAA 630X | Improvement |
| 012-101 | JAK2_V617F | | Stable |
| 020-102 | CALR_52b_del | | Increase |
| 021-101 | CALR_KKRK374X | | Stable |
| 022-101 | JAK2_V617F | | Improvement |
| 030-101 | JAK2_V617F | EZH2_F120X GPR183_T81I | Stable |
| 032-101 | JAK2_V617F | ASXL1_-642X | Improvement |

The following table presents examples of changing VAFs for patients in the study.

| Patient | Day | Mutation(s) | Diagnosis | Outcome |
|---|---|---|---|---|
| 010-104 | 91 | SF3B1_K700E | 26.37 | |
| | | DNMT3A_V687G | 94.96 | Probable DNMT3A CHIP followed by TET2 |
| | | JAK2_V617F | 27.02 | followed by JAK2/SF3B1 - only |
| | | TET2_S1284F | 45.72 | JAK2/SF3B1 -bearing clone is reduced; |
| | | | | treatment associated with dramatic |
| | | | | improvement of Hb and normalized platelet |
| | | | | count. |
| 011-105 | 182 | ASXL1_HHCHREAA630X | 21.48 | Disproportionate reduction of JAK2 clone |
| | | JAK2_V617F | 41.45 | compared to ASXL1; significant |
| | | | | improvement in Hb, spleen volume, and |
| | | | | WBC count. |
| 008-101 | 112 | MPL_W515K | 94.64 | ASXL1 clone is reduced while homozygous |
| | | ASXL1_Q780* | 18.58 | MPL virtually unaffected; good clinical |
| | | | | response but Day 84 washout rebound was |
| | | | | discouraging - withdrew consent. (Washout |
| | | | | later eliminated.) |
| 008-103 | 570 | CALR_K385NCX | 22.42 | ASXL1 clone reduced while the CALR clone |
| | | ASXL1_R693* | 29.22 | has homozygosed - excellent clinical |
| | | | | improvement for first year but spleen |
| | | | | volume improvement waned. Went to |
| | | | | transplant. |

From the foregoing description, one skilled in the art can easily ascertain the essential characteristics of this invention, and without departing from the spirit and scope thereof, can make various changes and modifications of the invention to adapt it to various usages and conditions.

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

All references cited in this specification are hereby incorporated by reference. The discussion of the references herein is intended merely to summarize the assertions made by their authors and no admission is made that any reference constitutes prior art relevant to patentability. Applicant reserves the right to challenge the accuracy and pertinence of the cited references.

1. A method of treating a myeloproliferative neoplasm, comprising administering to a subject in need thereof an amount of N-[(2S)-5-{[(1R, 2S)-2-(4-fluorophenyl) cyclopropyl]amino}-1-(4-methylpiperazin-1-yl)-1-oxopentan-2-yl]-4-(1H-1,2,3-triazol-1-yl)benzamide, bis-tosylate salt 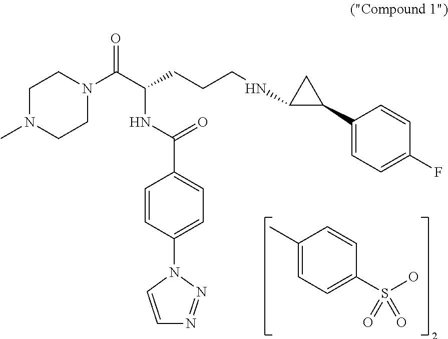
sufficient to maintain in the subject a platelet count of about 50×10⁹ to about 100×10⁹ platelets/L.

2. (canceled)

3. (canceled)

4. (canceled)

5. (canceled)

6. (canceled)

7. (canceled)

8. (canceled)

9. A method for maintaining the bone marrow blast count or reducing the bone marrow blast count to <5% in a subject in need thereof, the method comprising administering an amount of N-[(2S)-5-{[(1R, 2S)-2-(4-fluorophenyl) cyclopropyl]amino}-1-(4-methylpiperazin-1-yl)-1-oxopentan-2-yl]-4-(1H-1,2,3-triazol-1-yl)benzamide, bis-tosylate salt 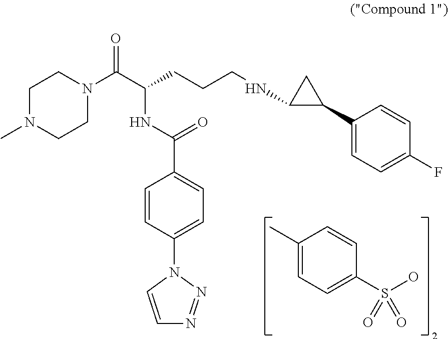
sufficient to maintain in the subject a platelet count of about 50×10⁹ to about 100×10⁹ platelets/L.

10. The method of claim 9, wherein the method suppresses

11. The method of claim 10, wherein the method reduces reducing the malignant cell burden measured by the mutant allele frequency of myeloid cells in the subject in need thereof.

12. The method of claim 10, wherein the method eliminates malignant myeloid cells in the subject in need thereof (“Compound 1”) sufficient to maintain in the subject a platelet count of about 50×10⁹ to about 100×10⁹ platelets/L.

13. A method for reducing reticulin and collagen bone marrow fibrosis in a subject in need thereof, comprising administering to the subject an amount of N-[(2S)-5-{[(1R, 2S)-2-(4-fluorophenyl) cyclopropyl]amino}-1-(4-methylpiperazin-1-yl)-1-oxopentan-2-yl]-4-(1H-1,2,3-triazol-1-yl)benzamide, bis-tosylate salt 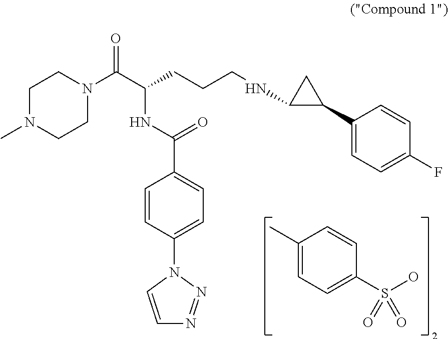
sufficient to maintain in the subject a platelet count of about 50×10⁹ to about 100×10⁹ platelets/L.

14. (canceled)

15. (canceled)

16. The method of claim 10, wherein the method reduces mutant allele burden in the subject in need thereof.

17. The method as recited in claim 16, wherein said mutant allele is an allele of one or more genes chosen from Janus Kinase 2 (JAK2), myeloproliferative leukemia virus oncogene (MPL) and calreticulin (CALR).

18. A method for reducing a pathologically elevated red blood cell mass in a subject in need thereof, the method comprising administering to the subject an amount of N-[(2S)-5-{[1R, 2S)-2-(4-fluorophenyl) cyclopropyl]amino}-1-(4-methylpiperazin-1-yl)-1-oxopentan-2-yl]-4-(1H-1,2,3-triazol-1-yl)benzamide, bis-tosylate salt 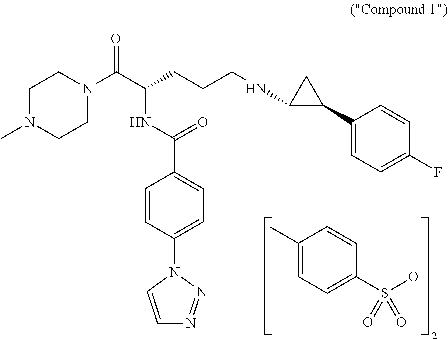
sufficient to maintain in the subject a platelet count of about 50×10⁹ to about 100×10⁹ platelets/L.

19. The method as recited in claim 18, wherein said subject has polycythemia vera.

20. The method as recited in claim 18, wherein the elevated blood cell mass is measured as hematocrit or blood hemoglobin.

21. The method as recited in claim 20, wherein measured blood hemoglobin has a value greater than 16.5 g/dL for a male subject or greater than 16.0 g/dL for a female subject.

22. The method as recited in claim 20, wherein measured hematocrit is greater than 49% for a male subject or greater than 48% for a female subject.

23. The method as recited in claim 18, wherein the elevated blood cell mass is measured by isotopic red cell mass measurement.

24. The method as recited in claim 23, wherein the increased red cell mass is greater than 25% above mean normal predicted value.

25. The method of claim 10, wherein the method reduces the mass of malignant myeloid cells in the subject in a subject in need thereof.

26. (canceled)

27. A method for reducing abnormal spleen size or volume in a subject in need thereof, the method comprising administering to the subject an amount of N-[(2S)-5-{[(1R, 2S)-2-(4-fluorophenyl) cyclopropyl]amino}-1-(4-methylpiperazin-1-yl)-1-oxopentan-2-yl]-4-(1H-1,2,3-triazol-1-yl)benzamide, bis-tosylate salt 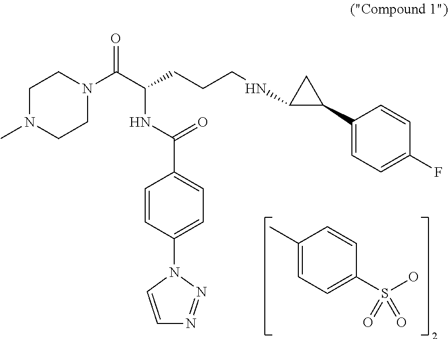
sufficient to maintain in the subject a platelet count of about 50×10⁹ to about 100×10⁹ platelets/L.

28. (canceled)

29. (canceled)

30. (canceled)

31. A method for reducing the frequency of thrombosis and hemorrhage in a subject in need thereof, the method comprising administering an amount of N-[(2S)-5-{[(1R, 2S)-2-(4-fluorophenyl) cyclopropyl]amino}-1-(4-methylpiperazin-1-yl)-1-oxopentan-2-yl]-4-(1H-1,2,3-triazol-1-yl)benzamide, bis-tosylate salt 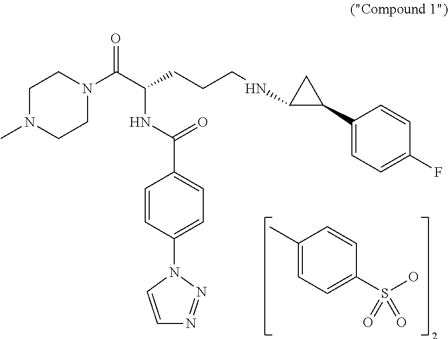
sufficient to maintain in the subject a platelet count of about 50×10⁹ to about 100×10⁹ platelets/L.

32. A method for reducing the constitutional symptoms of myelofibrosis measured by a subject-reported survey in a subject having myelofibrosis, the method comprising administering to the subject an amount of N-[(2S)-5-{[1R, 2S)-2-(4-fluorophenyl) cyclopropyl]amino}-1-(4-methylpiperazin-1-yl)-1-oxopentan-2-yl]-4-(1H-1,2,3-triazol-1-yl)benzamide, bis-tosylate salt 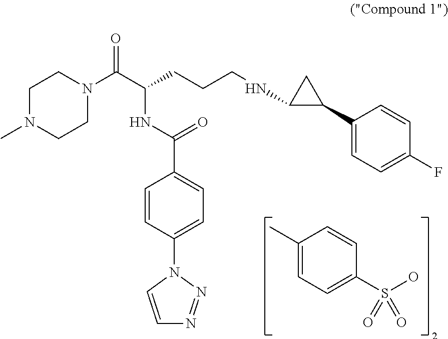
sufficient to maintain in the subject a platelet count of about 50×10⁹ to about 100×10⁹ platelets/L.

33. The method as recited in claim 32, wherein said constitutional symptoms comprise one or more symptoms chosen from fatigue, early satiety, abdominal discomfort, inactivity, problems with concentration, numbness and/or tingling in the hands and feet, night sweats, pruritis, bone pain, fever greater than 100° F., and unintentional weight loss.

34. The method as recited in claim 32, wherein said subject-reported survey is the Myeloproliferative Neoplasm Assessment Form Total Symptom Score (MPN-SAF:TSS).

35. The method as recited in claim 34, wherein one or more symptoms is reduced by at least 50% in its ranking on the MPN-SAF:TSS score.

36. The method as recited in claim 1, wherein the subject in need has a myeloproliferative neoplasm.

37. The method as recited in claim 36, wherein the myeloproliferative neoplasm is myelofibrosis (MF).

38. The method as recited in claim 37, wherein the myelofibrosis is chosen from primary myelofibrosis (PMF), post-PV myelofibrosis (PPV-MF), and post-ET myelofibrosis (PET-MF).

39. The method as recited in claim 38, wherein the myelofibrosis is primary myelofibrosis (PMF).

40. The method as recited in claim 36, wherein the myeloproliferative neoplasm is polycythemia vera (PV).

41. The method as recited in claim 36, wherein the myeloproliferative neoplasm is essential thrombocythemia (ET).

42. The method as recited in claim 1, wherein said subject has, or the subject's malignant myeloid cells have, a mutation in one or more genes chosen from Janus Kinase 2 (JAK2), myeloproliferative leukemia virus oncogene (MPL) and calreticulin (CALR).

43. The method as recited claim 42, further comprising the step of determining whether said subject has mutations in one or more genes chosen from Janus Kinase 2 (JAK2), myeloproliferative leukemia virus oncogene (MPL) and calreticulin (CALR).

44. The method as recited in claim 1, wherein the amount of Compound 1 is sufficient to maintain in the subject a platelet count of about 50×10⁹ to about 75×10⁹ platelets/L.

45. The method as recited in claim 1, wherein the amount of Compound 1 is about 0.5 mg/kg/d to about 1.5 mg/kg/d.

46. The method as recited in claim 45, wherein the amount of Compound 1 is about 0.7 mg/kg/d to about 1.2 mg/kg/d.

47. The method as recited in claim 1, wherein the amount of Compound 1 is about 40 mg to about 100 mg per day.

48. The method as recited in claim 47, wherein the amount of Compound 1 is about 50 mg to about 85 mg per day.

49. The method as recited in claim 1, wherein the subject is administered a starting dose of 0.5 mg/kg/d Compound 1, then, after one week:
if platelet count is ≥90×10⁹ platelets/L and the % platelet reduction is <50% from previous visit, the subject's dose is adjusted to add 0.2 mg/kg/d Compound 1 to the daily dose;
if platelet count is ≥90×10⁹ platelets/L and the % platelet reduction is ≥50% from previous visit, the subject's dose is adjusted to add 0.1 mg/kg/d Compound 1 to the daily dose;
if platelet count is between 40×10⁹ platelets/L and 89×10⁹ platelets/L, the daily dose of Compound 1 is maintained;
if platelet count is between 25×10⁹ platelets/L and 39×10⁹ platelets/L, the subject's dose is adjusted to decrease the current mg/kg daily dose of Compound 1 by 25%;
if platelet count is <25×10⁹ platelets/L, withhold dosing until platelets return to >50×10⁹ platelets/L, then the subject's dose is adjusted to administer Compound 1 at 50% of the dose that was administered when platelet count fell below 25×10⁹ platelets/L; and
optionally, approximately weekly throughout the course of therapy, repeating the platelet count assessment and dose adjustment steps until the subject's platelet count is about 50×10⁹ to about 100×10⁹ platelets/L.

50. The method of claim 1, wherein the method of treating comprises
administering a starting dose of 0.5 mg/kg/d Compound 1;
after about one week, assessing the subject's platelet count;
if platelet count is ≥90×10⁹ platelets/L and the % platelet reduction is <50% from previous visit, add 0.2 mg/kg/d Compound 1 to the daily dose;
if platelet count is ≥90×10⁹ platelets/L and the % platelet reduction is ≥50% from previous visit, add 0.1 mg/kg/d Compound 1 to the daily dose;
if platelet count is between 40×10⁹ platelets/L and 89×10⁹ platelets/L, maintain the current daily dose of Compound 1;
if platelet count is between 25×10⁹ platelets/L and 39×10⁹ platelets/L, decrease the current mg/kg daily dose of Compound 1 by 25%;
if platelet count is <25×10⁹ platelets/L, withhold dosing until platelets return to >50×10⁹ platelets/L, then administer Compound 1 at 50% of the dose that was administered when platelet count fell below 25×10⁹ platelets/L; and
optionally, repeating the platelet count assessment and dose adjustment steps approximately weekly until the subject's platelet count is about 50×10⁹ to about 100×10⁹ platelets/L.

51. The method of claim 1, A wherein the subject has a mutant allele

52. The method as recited in claim 51, wherein said mutant allele is an allele of one or more genes chosen from Janus Kinase 2 (JAK2), such as JAK^{V617F}, myeloproliferative leukemia virus oncogene (MPL), such as MPL^{W515K}, and calreticulin (CALR), such as CALR^{52b_del}, CALR^{K385NCX}, or CALR^{KKRK374X}.

53. The method as recited in claim 51, wherein said mutant allele is an allele of one or more genes chosen from chosen from DNMT3A, IDH1/2, TET2, ASXLI, EZH2, TP53, NF1, NRAS, KRAS, SF3B1, U2AF1, SRSF2, RUNX1, CBL, ZBTB33, PRPF8, CNTN5, FREM2, MAP1B, and GPR183.

54. (canceled)

55. The method as recited in claim 51, wherein said mutant allele is an allele of the gene Biorientation Of Chromosomes In Cell Division 1 Like 1 (BOD1L1).

56. (canceled)
